(19) United States
(12) Patent Application Publication
(10) Pub. No.: US 2008/0255244 A1
(43) Pub. Date: Oct. 16, 2008

(54) CaM Kinase II Inhibitor Improves Retinoic Acid Therapy and Inhibits the Proliferation of Myeloid Leukemia Cells

(75) Inventors: Steven J. Collins, Lake Forest Park, WA (US); Jutong Si, Seattle, WA (US)

Correspondence Address: MYERS BIGEL SIBLEY & SAJOVEC, PO BOX 37428, RALEIGH, NC, 27627, US

(73) Assignee: Fred Hutchinson Cancer Research Center

(21) Appl. No.: 12/099,347

(22) Filed: Apr. 8, 2008

Related U.S. Application Data
(60) Provisional application No. 60/911,121, filed on Apr. 11, 2007.

(51) Int. Cl. A61K 31/07; A61P 35/00

(52) U.S. Cl. 514/725

(57) ABSTRACT

A method of treating a cancer (e.g., myeloid leukemia) in a subject in need thereof, is carried out by administering the subject a CaM kinase II (CaMK II) inhibitor. In some embodiments, the CaMK II inhibitor is administered concurrently with a retinoid. In some embodiments, the CaMK II inhibitor is CaMK II gamma inhibitor. Compositions and formulations useful for carrying out such methods are also described.

### RELATED APPLICATIONS

This application claims the benefit of U.S. Provisional Patent Application Ser. No. 60/911,121, filed Apr. 11, 2007, the disclosure of which is incorporated by reference herein in its entirety.

### GOVERNMENT SUPPORT

This invention was made with Government Support under Grant Nos. HL54881 and RO1 CA118971 from the National Institutes of Health. The United States Government has certain rights to this invention.

### FIELD OF THE INVENTION

This invention concerns methods and compositions for the treatment of cancer, particularly leukemias such as acute myeloid leukemia.

### BACKGROUND OF THE INVENTION

Hematopoiesis involves an intricate, functional interaction between lineage-specific cytokines and lineage-specific transcription factors. Among these transcription factors the retinoic acid (RA) receptors (RARs) are important regulators of mycloid lineage differentiation. These receptors are members of the ligand-activated nuclear receptor (NR) family and include two distinct families, the RARs and RXRs, which bind as RAR-RXR heterodimers to their specific target sequences, the retinoic acid response elements (RAREs). RARs play a critical role in regulating myeloid differentiation since RA stimulates the granulocytic differentiation of normal hematopoietic precursors (Gratas, C. et al., Leukemia 7:1156-1162 (1993)), and knock-out mice deficient in both RARα and RARγ exhibit a block to granulocyte differentiation (Labrecque, J. et al., Blood 92:607-615 (1998)). Moreover, a critical genetic event in the development of human acute promyelocytic leukemia (APL) involves the acquisition of a t(15;17) chromosome translocation resulting in the leukemogenic PML-RARα fusion protein that acts as a dominant negative RAR to block normal myeloid differentiation (He, L.-Z. et al., Nature Genetics 18:126-135 (1998); Lin, R. et al., Nature 391:811-814 (1998)). This differentiation block can be overcome with relatively high, pharmacologic concentrations of RA, and the clinical use of RA to induce terminal differentiation of leukemia cells has had a remarkably beneficial impact on the therapy of APL (Huang, M. et al., Blood 72:567-574 (1988); Tallman, M. S. et al., Blood 99:759-767 (2002)).

Ligand binding to RAR/R alters its transcriptional activity by triggering a conformational change in this complex that inhibits its interaction with transcriptional corepressors while enhancing interaction with different transcriptional coactivators (Heery, D. M. et al., Nature 387:733-736 (1997); Glass, C. K., and Rosenfeld, M. G., Genes Dev 14:121-141 (2000)). A signature LxxLL binding motif or closely related sequences are present in the receptor interaction domains of a number of different nuclear hormone receptor coactivators including the SRC/p160 family (Torchia, J. et al., Current Opinion in Cellular Biology 10(3):373-383 (1998)) the ASC-2 coactivator (Lee, S. K. et al., Mol Endocrinol 15:241-254 (2001)) and the mediator complex TRAP220/DRIP205/MED1 subunit (Rachez, C. et al., Mol Cell Biol 20:2718-2726 (2000; Ren, Y. et al., Mol Cell Biol 20:5433-5446 (2000). This motif mediates the recruitment of these coactivators into the nuclear receptor transcription complex by directly contacting the AF-2 domain of the nuclear receptors. The transcriptional activity of the RARs is also regulated by protein kinase-mediated phosphorylation. Both protein kinase A (PKA) and cdk7 phosphorylate and enhance RAR transcriptional activity (Rochette-Egly, C. et al., Mol Endocrinol 9:860-871 (1995); Rochette-Egly, C. et al., Mol Endocrinol 9:860-871 (1995)), and diminished cdk7 phosphorylation of RAR with reduced RAR activity is observed in the genetic disorder, Xeroderma pigmentosum (Keriel, A. et al., Cell 109:125-135 (2002)).

The Ca⁺⁺/calmodulin-dependent protein kinases (CaMKs) are multifunctional Ser/Thr kinases whose activity is regulated through Ca⁺⁺ signaling (Hook, S. S. et al., Annu Rev Pharmacol Toxicol 41:471-505 (2001)). The most widely studied CaMKs include CaMKI, II and IV. In general CaMK activation is triggered by the binding of Ca⁺⁺/calmodulin (Ca⁺⁺/CaM), and the levels of Ca⁺⁺/CaM are regulated by changes in intracellular Ca⁺⁺ concentration. The CaMKs regulate the development and activity of multiple different cell types. For example, CaMKII and IV regulate cytokine expression in activated T-lymphocytes (Anderson, K. A., and Means, A. R., Mol Cell Biol 22:23-29 (2002); Nghiem, P. et al., Nature 371:347-350 (1994)). CaMKII also regulates dendritic morphogenesis through phosphorylation of the NeuroD transcription factor (Gaudilliere, B. et al., Neuron 41:229-241 (2004)). Moreover CaMKII, which comprises approximately 1-2% of total brain protein, also phosphorylates proteins involved in strengthening synaptic transmission, and this likely regulates learning and memory (reviewed in Lisman, J. et al., Nat Rev Neurosci 3:175-190 (2002)).

### SUMMARY OF THE INVENTION

A first aspect of the present invention is a method of treating a cancer (e.g., myeloid leukemia) in a subject in need thereof, comprising concurrently administering said subject a retinoid and a CaM kinase II (CaMK II) inhibitor. The CaMK II inhibitor administered in an amount effective to enhance the activity of said retinoid; and the said retinoid administered in an amount effective to treat the cancer). In some embodiments, the retinoid and the CaMK II inhibitor are administered in a synergistically effective amount. In some embodiments, the CaMK II inhibitor is CaMK II gamma inhibitor.

A second aspect of the present invention is a method of treating a cancer (e.g., myeloid leukemia) in a subject in need thereof, comprising administering said subject a CaM kinase II gamma inhibitor in a treatment effective amount.

A further aspect of the present invention is a pharmaceutical composition comprising, in combination, a CaMK II inhibitor and a retinoid. In some embodiments, the CaMK II inhibitor and said retinoid are included in said composition in a synergistically effective amount.

A further aspect of the present invention is the use of a CaMK II inhibitor, and/or a retinoid, for the preparation of a medicament for carrying out a method of treatment as described herein.

The foregoing and other objects and aspects of the present invention are explained in detail in the drawings and specification set forth below.

### BRIEF DESCRIPTION OF THE DRAWINGS

FIG. 1. CaM kinase isoform expression in hematopoietic cells. (A) The activity of an ATRA-responsive luciferase reporter (βRARE-tk-Luc, 25 μg) was assessed in electroporated HL60 cells six hours after treatment with the indicated concentrations of ionomycin and ATRA. (B) The activity of the luciferase reporter (βRARE-tk-Luc, 25 μg) was assessed in HL60 six hours after treatment with KN62 (5 uM), KN93 (5 uM) and ATRA (1 uM). (C) Western blots were performed utilizing the indicated antibodies. F9 (column 6) is a mouse embryonal carcinoma cell line. BaF3 (column 7) is a cultured pre-B cell line. BM cells (column 8) are kit-ligand dependent normal murine hematopoietic precursors. PT67 murine fibroblasts individually transduced with either rat CaMKIIα, human CaMKIα or human CaMKIV (column 9) together with brain lysates (column 1) serve as positive controls for the respective antibodies. (D) CaMK assays were performed on HL-60 cell lysates immunoprecipitated with the indicated antibodies.

FIG. 2. Regulation of RA receptor activity by CaMKII. (A) HL60 cells were electroporated with the βRARE-tk-Luc reporter (25 μg) together with expression vectors (20 μg) harboring wild type or constitutively activated (CA) CaMKII cDNAs (CaMKIIα). ATRA (1 μM) was added, and relative luciferase activity was determined in the cultured cells after six hours. (B) HL60 cells were stably transduced with empty (control) retroviral vectors and with vectors harboring CaMKIIγ shRNAs constructed as detailed in the Materials and Methods. Westerns identified six CaMKIIγ shRNAs transduced HL60 subclones (A6, B2, B5, B6, C2, C18) expressing reduced CaMKIIγ protein expression. (C) Pooled subclones of the shCaMKIIγ vector-transduced HL60 cells that exhibit reduced CaMKIIγ expression on Western blots (FIG. 2B) together with control (empty) vector transduced HL60 were lysed, immunoprecipitated with CaMKII antibody and the immunoprecipitates assayed for both Ca⁺⁺/calmodulin independent (EGTA) and Ca⁺⁺/calmodulin dependent (Ca⁺⁺/CaM) enzyme activity. (D) Pooled subclones of CaMKIIγ shRNA vector-transduced HL-60 cells that exhibit reduced CaMKIIγ expression/activity (FIG. 2B,C) as well as control (empty) vector transduced cells were electroporated with the βRARE-tk-Luc reporter (25 μg), and relative luciferase activity was determined six hours after the addition of ATRA (1 μM). (E) The subclones of the CaMKIIγ shRNA vector-transduced HL-60 cells that exhibit reduced CaMKIIγ expression/activity (FIG. 2B,C) as well as control (empty) vector transduced cells were treated with RA (1 nM) for five days followed by FACS-quantitation of Cd11b surface antigen. The displayed results represent the ratio of Cd11b expression in RA-induced vs uninduced cells utilizing six different subclones in three independent experiments. (F) HL60 cells were electroporated with the βRARE-tk-Luc reporter (25 μg) together with a control empty vector or a vector harboring the CaMKII inhibitory protein (CaMKIINα), and relative luciferase activity was determined six hours after the addition of ATRA (1 μM).

FIG. 3. CaMKII directly interacts with RARα through an LxxLL binding motif. (A) HL60 cell lysates treated with ATRA (1 μM) for the indicated time were immunoprecipitated with the indicated antibodies followed by Western blot analysis. (B) In vitro translated ³⁵S-labeled CaMKIIα was incubated with GST or a GST-RARα fusion protein attached to glutathione beads. The beads were washed and then subjected to SDS-PAGE. (C) Amino acid sequence indicating in bold the conserved LxxLL motif in CaMKII. All four CaMKII isoforms (α,β,γ,δ) harbor this identical sequence. In vitro translated ³⁵S-labeled CaMKII cDNAs harboring the indicated mutations engineered within this LxxLL motif were subjected to GST pulldown assays utilizing the GST-RARα fusion protein. (D) HL60 cells were electroporated with the βRARE-tk-Luc reporter (15 μg) together with expression vectors harboring parental CaMKIIα (WT) or CaMKIIα that harbors the indicated mutant LxxLL motif (LxxAA). Following six hours of incubation with ATRA (1 μM), relative luciferase activity was determined on cell extracts.

FIG. 4. Phosphorylation of RARα by CaMKIIγ. (A) In vitro translated RARα was antibody-purified and then subjected to an in vitro kinase reaction with in vitro translated CaMKII. (B) The four consensus CaMK sites (R-x-x-S/T) within the ligand binding domain of RARα are arbitrarily labeled 1-4. The (K-x-x-S/T) CaMK consensus site within the RARα hinge domain is labeled site 0. (C) A Gst-RARα fusion protein was constructed that harbors nonphosphorylatable alanine mutations (m) at consensus CaMK sites 1-4 (Gst-RARm(1,2,3,4)). This Gst-RARα fusion protein as well as the parental wild type Gst-RARα were subjected to in vitro kinase reactions utilizing CaMKIIγ immunoprecipitated from HL60 cells. The more intense lower band corresponds to the immunoprecipitated CaMKIIγ that is autophosphorylated during this in vitro kinase reaction. (D) A Gst-RARα fusion fragment harboring RARα amino acids 135-291 (RAR-Wt) as well as the same fusion protein that is mutated (T→A) at RARα site 0 (RAR-T209A,T210A) were subjected to an in vitro kinase reaction utilizing immunoprecipitated HL60 CaMKIIγ. The radiolabeled upper bands represent CaMKIIγ that is autophosphorylated during this reaction. (E) 293 cells were transfected with the indicated expression vectors and then metabolically labeled with [³²P] orthophosphate. After four hours anti-HA immunoprecipitates were electrophoresed on an agarose gel followed by radioautography. CaMKIIca is the constitutively active CaMKIIα. Anti-HA Western blots (lower row) served as a control for the amount of immunoprecipitated HA-RAR from each transfectant. (F) HL60R cells were electroporated with the βRARE-tk-LUC reporter (15 μg) together with expression vectors harboring the wild type (WT) or the indicated CaMKII (T209,T210) site-mutated RARα. Six hours after RA addition (1 μM) relative luciferase activity was determined on cell extracts.

FIG. 5. CaMKII phosphorylation of RARα enhances its interaction with transcriptional corepressors. (A) In the mammalian two hybrid, the activity of the transfected (UAS)₅ luciferase reporter is proportional to the interaction of Gal-N-CoR with RARαVP16. (B-D) Relative luciferase activity of the transfected (UAS)₅ luciferase reporter was determined in lysates of cultured MPRO promyelocytes co-transfected with the indicated Gal-NCoR and VP16 RARα wild type vs VP16 RARα mutated expression vectors. Cells were then treated with the indicated concentration of ATRA for six hours. In (B) KN62 treatment was for 48 hours. (E) FLDS cells that were stably transduced with the indicated HA-tagged wild type (WT) RARα and CaMKII site-mutated RARα (T209A,T210A) were treated with ATRA (1 μM) as indicated, and pulldowns from these cell lysates utilizing the Gst-NCoR fusion protein were subjected to anti-HA Western blot analysis.

FIG. 6. Retinoic acid induces changes in the association of CaMKIIγ with the C/EBPε promoter RARE. (A) Western blot of cell lysates exhibiting enhanced C/EBPε expression in ATRA-treated (1 uM) HL60. (B) The human C/EBPε 5′ region indicating the location of the ChIP PCR primers. (C) ChIP assays. HL60 cell chromatin from cells treated with ATRA (1 uM) for the indicated time was immunoprecipitated with the indicated antibodies, the DNA isolated and subjected to PCR utilizing the indicated C/EBPε promoter primer pairs.

FIG. 7. KN-62 regulates the differentiation of myeloid leukemia cell lines. (A) Wright Giemsa-stained HL-60 cells either (a) uninduced or treated for five days with (b) KN62 or (c) ATRA. (d) CD11b expression in HL-60 cells after five days exposure to KN62 (5 μM) or ATRA (1 μM). (B) Wright-Giemsa stained NB4 cells treated for five days with KN-62 (5 μM) and ATRA as indicated. Uninduced NB4 are primarily immature myeloblasts (a) and their differentiation is not significantly altered by KN62 treatment alone (b). NB4 cells treated with low concentration ATRA (10⁻⁹ M) display little differentiation (c) but granulocytic differentiation is markedly enhanced with the addition of KN62 (5 μM) (d). (C) Cd11b expression of NB4 cells treated for five days with the indicated concentrations of RA and/or KN-62. (D) Western blots with the indicated antibodies were performed on HL60 cell lysates following incubation with KN62 for the indicated period of time. (E) Western blots of NB4 cell lysates following a five day incubation with the indicated compounds.

FIG. 8. CaMKIIγ activation in myeloid leukemia cells. (A) Cell lysates from the indicated human leukemia cell lines were subjected to Western blots utilizing the indicated CaMKII antibodies. Antibodies to the catalytic subunit of PP2A (PP2Ac) serve as a loading control. (B) Cell lysates from primary AML samples were subjected to Western blots utilizing the indicated CaMKII antibodies. Antibodies to PP2Ac serve as a loading control. (C) RT-PCR was performed on RNA extracted from the indicated leukemia cell lines and normal CD34+ cells and the products were displayed on an agarose gel. The location of the sense (S) and antisense (AS) primers in relationship to the catalytic, regulatory, variable and association domains of the human CaMKIIγ coding sequence are indicated.

FIG. 9. Terminal differentiation/apoptosis of myeloid leukemia cells is associated with decreased CaMKIIγ activation. (A) Lysates from the HL60, NB4 and U937 myeloid cell lines treated with ATRA (1 μM) for the indicated period of time were subjected to Western blots. (B) Lysates from the indicated myeloid cell lines treated for five days with the indicated concentration of ATRA were subjected to Western blots. (C) Lysates from HL60 cells treated with TPA (0.1 μM) for the indicated time were subjected to Western blots. (D) Lysates from NB4 cells treated with arsenic trioxide (1 uM) for the indicated time were subjected to Western blots.

FIG. 10. Inhibiting bcr-abl activity/expression inhibits CaMKIIγ activation. (A) Lysates from K562 cells undergoing growth arrest following treatment with gleevec (5 uM) for the indicated times were subjected to Western blots. (B) Lysates from TonB210.1 cells depleted of doxycycline for the indicated period of time were subjected to Western blots. (C) IL-3 dependent TonB210.1 cells were deprived of IL-3 for 24 hours (lane 1). The cells were then treated with the indicated compounds simultaneously with the addition of doxycycline (lanes 2-8). After 24 hours cell lysates were harvested and subjected to Western blots with the indicated CaMKII antibodies. (D) K562 cells were cultured for 48 hours with the indicated chemical inhibitors of specific signal transduction pathways. Cell lysates were harvested and subjected to Western blot analysis.

FIG. 11. CaMKII inhibitors inhibit myeloid leukemia cell proliferation. (A) K562 cells were seeded in liquid suspension culture at 5×10⁴ cells/ml in the presence or absence of the indicated concentration of compounds, and cell counts were obtained at the indicated time. (B) The indicated myeloid leukemia cell lines were seeded in liquid suspension culture at 5×10⁴ cells/ml in the presence or absence of KN93, and cell counts obtained at the indicated time. (C) K562 cells were electroporated with the LXSN expression vector harboring the kinase dead, Lys⁴³ mutated, truncated CaMKII construct (kdCaMKII) as well as with the control (empty) LXSN vector. The electroporated cells were diluted into 96 well plates in liquid suspension culture in the presence of G418 (1 mg/ml). Following eight days of culture the total number of discrete, actively proliferating G418 resistant colonies within individual wells was determined.

FIG. 12. CaMKII regulates multiple signal transduction pathways in myeloid cells. (A,B) Lysates from K562 cells cultured for the indicated time in KN93 were subjected to Western blots. (C) The K562 cells transduced as described in Materials and Methods with either (i) the doxycycline (DOX) inducible expression vector/transactivator harboring the full length, Lys⁴³ mutated, kinase dead CaMKIIγ (kdCaMKIIγ (Tet-on)) or (ii) the CaMKII shRNA generating plasmids, were cultured in liquid suspension in the presence or absence of DOX as indicated, and cell counts obtained after 48 hours. (D) Lysates from K562 cells transduced with the indicated vectors were subjected to Western blots utilizing the indicated antibodies. For lanes 2 and 4 doxycycline (DOX) treatment was for two days.

FIG. 13. Stat3 is directly phosphorylated and activated by CaMKIIγ at Ser727. (A, i). Cell lysates from the indicated human leukemia cell lines were subjected to Western blots utilizing the indicated Stat3 antibodies. ii). Cell lysates from the same primary AML samples depicted in FIG. 1B were subjected to Western blots utilizing the indicated Stat3 antibodies. B). The IL-3 dependent TonB210.1 cells were deprived of IL-3 for 24 hours. The cells were then treated with the indicated compounds immediately prior to the addition of doxycycline. After an additional 24 hours cell lysates were harvested and subjected to Western blots with the indicated Stat3 antibodies. C). K562 cells were electroporated with a luciferase reporter driven by a Stat3 response element. The indicated concentrations of KN93 and the Stat 3 inhibitor, cucurbitacin I, were added and relative luciferase activity determined on cell lysates following 48 hours of culture. D, i) K562 cell lysates were immunoprecipitated with control IgG or a Stat3 antibody. The immunoprecipitates were then subjected to Western blot analysis with the indicated antibodies. ii,) A bacterial—expressed GST-Stat3 fusion protein was incubated in vitro with CaMKIIγ that had been immunoprecipitated from HL60 cells. Western blots were then performed on the reaction mixtures utilizing the indicated Stat3 antibodies. iii,). NIH3T3 cells were transfected with the empty LXSN expression vector (lane 1), the same vector harboring the coding sequences of a constitutively active (ca) CaMKIIγ (lane 2), or vector harboring the kinase dead (kd) CaMKIIγ (lane 3). After 48 hours Western blots were performed on cell lysates utilizing the indicated Stat3 antibodies.

### DETAILED DESCRIPTION OF THE PREFERRED EMBODIMENTS

The term “treat” as used herein refers to any type of treatment that imparts a benefit to a patient afflicted with a disease, including improvement in the condition of the patient (e.g., in one or more symptoms), delay in the progression of the disease, etc.

The term “pharmaceutically acceptable” as used herein means that the compound or composition is suitable for administration to a subject to achieve the treatments described herein, without unduly deleterious side effects in light of the severity of the disease and necessity of the treatment.

As used herein, the administration of two or more compounds “in combination” means that the two compounds are administered closely enough in time that the presence of one alters the biological effects of the other. The two compounds may be administered simultaneously (i.e., concurrently) or sequentially. Simultaneous administration may be carried out by mixing the compounds prior to administration, or by administering the compounds at the same point in time but at different anatomic sites or using different routes of administration.

“Cancer” as used herein refers to any type of cancer, including but not limited to acute myelogenous leukemia, chronic myelogenous leukemia, gastrointestinal stromal tumor, small cell lung cancer, non-small cell lung cancer, ovarian cancer, melanoma, mastocytosis, germ cell tumors, pediatric sarcomas, breast cancer, colorectal cancer, pancreatic cancer, prostate cancer, etc.

The present invention is primarily concerned with the treatment of human subjects, but the invention may also be carried out on animal subjects, particularly mammalian subjects such as mice, rats, dogs, cats, livestock and horses for veterinary purposes, and for drug screening and drug development purposes.

The disclosures of all United States patent references cited herein are to be incorporated by reference herein in their entirety.

### 1. Active Compounds.

CaM Kinase II Inhibitors. “CAMKII inhibitor” as used herein may be any suitable compound with the desired biological activity. Numerous examples of CaMKII inhibitors are known. Examples include calcium chelators, calmodulin antagonists, small peptides based on CaMKII protein sequence, nucleic acid-based inhibitors, and mixtures thereof. Particular examples include, but are not limited to, KN62, KN93, H89, HA1004, HA1077, autocamtide-2 related inhibitory peptide or a myristoylated form thereof, K-252a, staurosporine, lavendustin C, BAPTA tetrasodium salt, 5,5′-dibromo-BAPTA, tetrasodium salt, BAPTA/AM, 5,5′-difluoro-BAPTA/AM, EDTA tetrasodium salt, EGTA, EGTA/AM, MAPTAM, TPEN, calmidazolium chloride, calmodulin binding domain, chlorpromazine, Compound 48/80, fluphenazine-N-2-chloroethane dihydrochloride, melittin, ophiobolin A, pentamidine isethionate, phenoxybenzamine, trifluoperazine, W-5, W-7, W-12, W-13, CaM kinase II 290-309, [Ala286]CaMKII Inhibitor 281-301, CaMKII Inhibitor 281-309, and mixtures thereof. Other examples include, but are not limited to, a nucleic acid-based inhibitor capable of modulating expression of CaMKII and selected from the group consisting of an antisense polynucleotide, a ribozyme, RNAi, a triple helix polynucleotide, and mixtures thereof. See, e.g., Z. Wang, Method and Composition for Potentiating an Opiate Analgesic, US Patent Application Publication No. 20040220203; See also US Patent Application Publication Nos. 20070298999 and 20070071724. In some embodiments, inhibitors of CaMKII gamma are preferred.

Retinoids. Any suitable retinoid can be used as active agent for the present invention. Examples include but are not limited to retinoic acid (e.g., all-trans-retinoic acid, 13-cis-retinoic acid, 9-cis-retinoic acid) and derivatives thereof. Additional examples include, but are not limited to: (all-E)-9-(4-methoxy-2,3,6-trimethylphenyl)-3,7-dimethyl-2,4,6,8-nonatetraenoic acid, (E,E,E)-7-(2,3-dihydro-1,1,3,3-tetramethyl-1H-inden-5-yl)-3,7-dimethyl-2,4,6-octatrienoic acid, (E,E,E)-7-(5,6,7,8-tetrahydro-5,5,8,8-tetramethyl-2-naphthalenyl)-3,7-dimethyl-2,4,6-octatrienoic acid, (E)-4-[(2,3-dihydro-1,1,3,3-tetramethyl-1H-inden-5-yl)-1-propenyl]benzoic acid, (E)-4-[2-(5,6,7,8-tetrahydro-5,5,8,8-tetramethyl-2-naphthalenyl)-1-propenyl]benzoic acid, (E)-4-[2-(5,6,7,8-tetrahydro-3-methyl-5,5,8,8-tetramethyl-2-naphthalenyl)-1-propenyl]benzoic acid, 2-(5,6,7,8-tetrahydro-5,5,8,8-tetramethyl-2-naphthyl)-6-naphthalene carboxylic acid, (E)-4-[2-(5,6,7,8-tetrahydro-5,5,8,8-tetramethyl-2-naphthalenyl)-1-propenyl]benzene sulfonic acid, (E,E)-4-[2-methyl-4-(2,6,6-trimethyl-1-cyclohexen-1-yl)-1,3-butadienyl]benzoic acid, (E,E)-4-[4-methyl-6-(2,6,6-trimethyl-1-cyclohexen-1-yl)-1,3,5-hexatrienyl]benzoic acid, (E)-6-[2-(2,6,6-trimethyl-1-cyclohexen-1-yl)-ethenyl]-2-naphthalene carboxylic acid, (E)-4-[2-(5,6,7,8-tetrahydro-8,8-dimethyl-2-naphthalenyl)-1-propenyl]benzoic acid, 4-[(5,6,7,8-tetrahydro-5,5,8,8-tetramethyl-2-naphthenyl)ethynyl]benzoic acid, (E)-4-[2-(5,6,7,8-tetrahydro-3-methyl-5,5,8,8-tetramethyl-2-naphthalenyl)-1-propenyl]benzoic acid, 4-(5,6,7,8-tetrahydro-5,5,8,8-tetramethyl-2-naphthalenylcarbamoyl)benzoic acid, 4-(5,6,7,8-tetrahydro-5,5,8,8-tetramethyl-2-naphthamido) benzoic acid, (E)-4-[3-(3,5-ditert-butylphenyl)-3-oxo-1-propenyl]benzoic acid, 6-[(5,6,7,8-tetrahydro-5,5,8,8-tetramethyl-2-naphthalenyl)ethynyl]3-pyridine carboxylic acid, 2-(5,6,7,8-tetrahydro-5,5,8,8-tetramethyl-2-naphthyl)-6-benzo(b) thiophene carboxylic acid, 4-(5,6,7,8-tetrahydro-5,5,8,8-tetramethyl-2-anthracenyl)benzoic acid, 6-[3-(1-adamantyl)-4-methoxyphenyl]-2-naphthoic acid, 4-[3-(1-adamantyl)-4-methoxybenzamido]benzoic acid, 4-[3-(1-adamantyl)-4-methoxy benzoylthio]benzoic acid, 4-[3-(1-adamantyl)-4-methoxy benzoyloxy]benzoic acid, 2-(5,6,7,8-tetrahydro-5,5,8,8-tetramethyl-2-naphthyl)-6-carbonyl naphthalene carboxylic acid,trans-(5,5,8,8-tetramethyl-5,6,7,8-tetrahydro-2-naphthyl)-4-carbonyl-alpha-methyl cinnamic acid, the vitamin A or retinol, as well as its esters, such as the acetate, propionate or palmitate of retinol, aldehyde of vitamin A or retinal 4-[3-(1-adamantyl)-4-methoxybenzoyloxy]-2-fluorobenzoic acid, 4-[3-(1-adamantyl)-4-methoxybenzoyloxy]-2-methylbenzoic acid, 4-[3-(1-adamantyl)-4-methoxybenzoyloxy]-2-hydroxybenzoic acid, 4-[5-(1-adamantyl)-2-fluoro-4-methoxybenzoyloxy]benzoic acid, 4-[3,5-di-tert.butyl-4-hydroxybenzoyloxy]benzoic acid, 4-[3-(1-adamantyl)-4-vinylbenzoyloxy]benzoic acid, 4-[3-(1-adamantyl)-4-ethylbenzoyloxy]benzoic acid, 4-[3-(1-adamantyl)-4-allyloxybenzoyloxy]benzoic acid, 4-[3-(1-adamantyl-4-methylthiobenzoyloxy]benzoic acid, 4-(5,6,7,8-tetrahydro-5,5,8,8-tetramethyl-2-naphthylglyoxyloyloxy)benzoic acid, 4-(5,6,7,8-tetrahydro-5,5,8,8-tetramethyl-2-naphthoyloxymethyl)benzoic acid, 4-(3,5-di-tert.butyl-4-hydroxybenzoyloxymethyl)benzoic acid, 4-(3-tert-butyl-4-methoxybenzoyloxymethyl)benzoic acid, 4-(5,6,7,8-tetrahydro-5,5,8,8-tetramethyl-2-naphthoylmethyloxy)benzoic acid, 4-[1-(5,6,7,8-tetrahydro-5,5,8,8-tetramethyl-2-naphthoyloxy)ethyl]benzoic acid, 4-[[(5,6,7,8-tetrahydro-5,5,8,8-tetramethyl-2-naphthyl)ethyloxy]carbonyl]benzoic acid, 4-(5,6,7,8-tetrahydro-5,5,8,8-tetramethyl-2-naphthoylmethylamino)benzoic acid, 4-(5,6,7,8-tetrahydro-5,5,8,8-tetramethyl-2-naphthoyl formamido)benzoic acid, 4-(.alpha.-hydroxy-5,6,7,8-tetrahydro-5,5,8,8-tetramethyl-2-naphthylacetamido) benzoic acid, 4-(.alpha.-fluoro-5,6,7,8-tetrahydro-5,5,8,8-tetramethyl-2-naphthylacetamido) benzoic acid, 6-[3-(1-adamantyl)-4-(2,3-dihydroxypropyloxy)phenyl]-2-naphthoic acid, 6-[3-(1-adamantyl)-4-(3-hydroxypropyloxy)phenyl]-2-naphthoic acid, 6-[3-(1-adamantyl)-4-acetoxymethylphenyl]-2-naphthoic acid, 6-[3-(1-adamantyl)-4-methoxycarbonylphenyl]-2-naphthoic acid, 6-[3-(1-adamantyl)-4-methoxycarbonylethylphenyl]-2-naphthoic acid, 6-[3-(1-adamantyl)-4-(2-hydroxypropyl)phenyl]-2-naphthoic acid, 2-hydroxy-4[2-hydroxy-2-(5,6,7,8-tetrahydro-5,5,8,8-tetramethyl-2-naphthyl)ethoxy]benzoic acid, methyl 2-hydroxy-4-[2-hydroxy-2-(5,6,7,8-tetrahydro-5,5,8,8-tetramethyl-2-naphthyl)ethoxy]benzoate, 2-hydroxy-4-[2-hydroxyimino-2-(5,6,7,8-tetrahydro-5,5,8,8-tetramethyl-2-naphthyl)ethoxy]benzoic acid, 2-acetyloxy-4-[2-acetyloxy-2-(5,6,7,8-tetrahydro-5,5,8,8-tetramethyl-2-naphthyl)ethoxy]benzoic acid, 2-hydroxy-4-[2-acetyloxy-2-(5,6,7,8-tetrahydro-5,5,8,8-tetramethyl-2-naphthyl)ethoxy]benzoic acid, 2-acetyloxy-4-[2-hydroxy-2-(5,6,7,8-tetrahydro-5,5,8,8-tetramethyl-2-naphthyl)ethoxy]benzoic acid, 4-(N-methyl-5,6,7,8-tetrahydro-5,5,8,8-tetramethyl-2-naphthylcarboxaminidino)benzoic acid, 4-(5,6,7,8-tetrahydro-5,5,8,8-tetramethyl-2-naphthylcarboxamidino)benzoic acid. Numerous particular examples are known, including but not limited to those described in U.S. Pat. Nos. 6,764,698, and 5,556,844, the disclosures of which are incorporated by reference herein in their entirety.

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

Active compounds can be administered in the form of pharmaceutically acceptable prodrugs. The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299 Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

### 2. Pharmaceutical Formulations.

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising a compound of Formula (I), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanolin, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to compounds of formula (I) or their salts, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

### 3. Dosage and Routes of Administration.

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any specific compound, the use of which is in the scope of present invention, will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the active compound, including the cases where a salt is employed. Toxicity concerns at the higher level may restrict intravenous dosages to a lower level such as up to about 10 or 50 mg/kg, with all weights being calculated based upon the weight of the active base, including the cases where a salt is employed. A dosage from about 10 mg/kg to about 50 or 100 mg/kg may be employed for oral administration. Typically, a dosage from about 0.5 mg/kg to 5 or 10 mg/kg may be employed for intramuscular or intravenous injection.

The present invention is explained in greater detail in the following non-limiting examples.

### Example 1

### CaM Kinase II Regulates Retinoic Acid Receptor Transcriptional Activity and the Differentiation of Myeloid Leukemia Cells

### Results

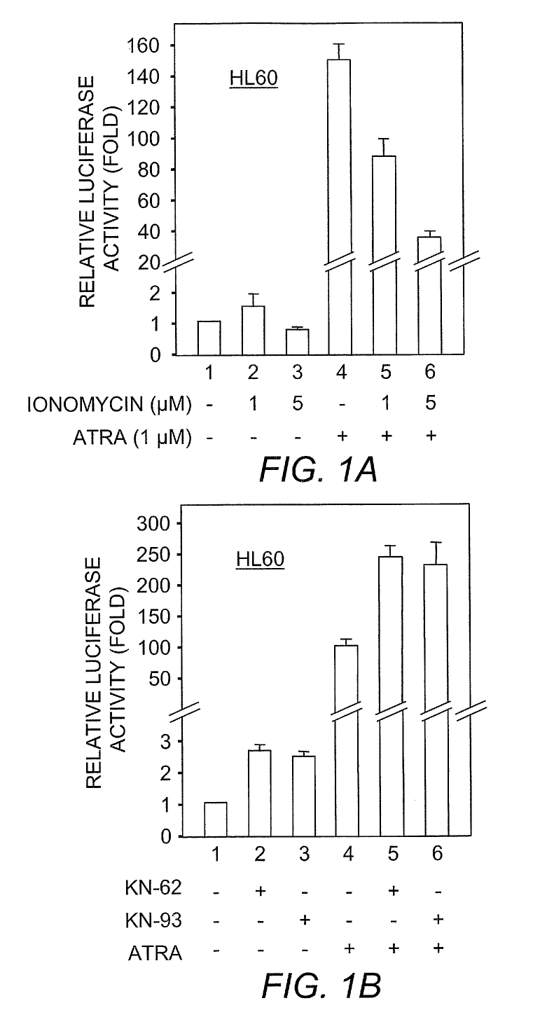

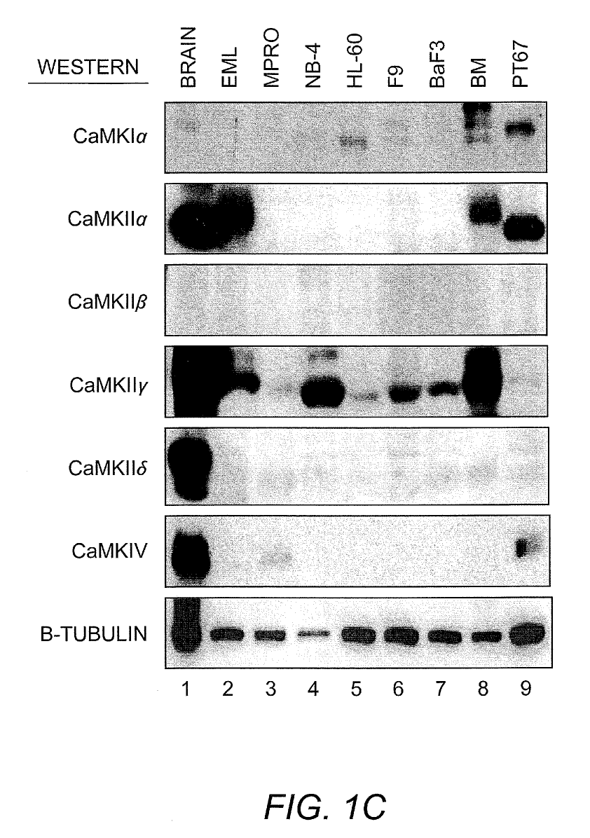

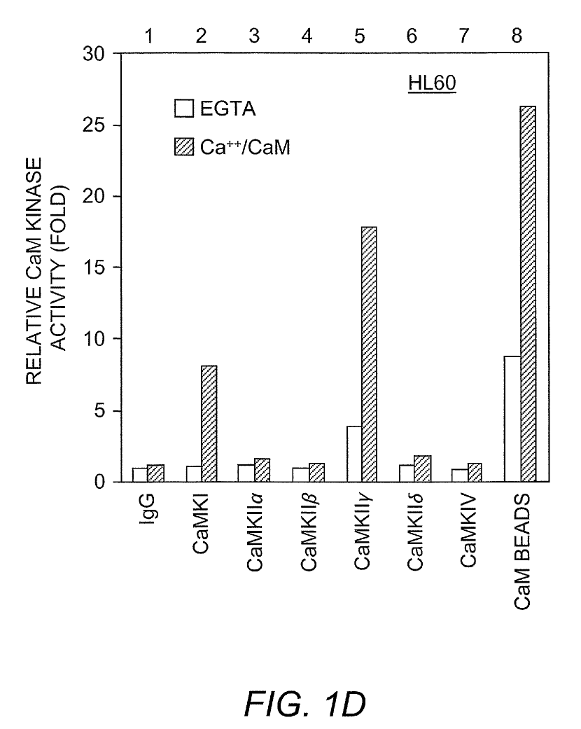

Expression and activity of the CaM kinases in differentiating myeloid cells. All-trans retinoic acid (ATRA) induces the terminal granulocytic differentiation of certain cultured hematopoietic cells, and we have been utilizing such cell lines including HL60 (Breitman, T. et al., Proc Natl Acad Sci USA 77:2936-2940 (1980)), NB4 (Lanotte, M. et al., Blood 77:1080-1086 (1991)) and MPRO (Tsai, S., and Collins, S., Proc. Natl. Acad. Sci. USA 90:7153-7157 (1993)) to define factors that regulate retinoic acid receptor (RAR) transcriptional activity in differentiating myeloid cells. We observed that the Ca⁺⁺ ionophore, ionomycin, inhibited the ATRA-induced activity of a retinoic acid response element (RARE)-driven luciferase reporter in HL60 myeloid cells (FIG. 1A, lanes 4-6). Such Ca⁺⁺ ionophores can potentially regulate many different Ca⁺⁺ dependent enzymes including the CaMKs. To assess whether the CaMKs are involved in regulating RAR transcriptional activity, we utilized KN-62 and KN-93, both potent small molecule inhibitors of the CaMKs (IC₅₀=0.9 μM), which directly interfere with the binding of Ca⁺⁺/CaM to the different CaMKs (Tokumitsu, H. et al., J Biol Chem 265:4315-4320 (1990); Sumi, M. et al., Biochem Biophys Res Commun 181:968-975 (1991)). We found that in HL60 myeloid cells, both KN-62 and KN-93 enhance RAR transcriptional activity (FIG. 1B). We also found similar results in other transfected myeloid cells including MPRO and NB4 (not shown). The observed enhancement of RAR activity by small molecule inhibitors of the CaMKs suggest that endogenous CaMKs are involved in downregulating the activity of the RARs in myeloid cells.

To further assess the role that the endogenous CaMKs might play in RAR activity and myeloid differentiation, we performed Western blots to compare the expression of the different CaMKs in different hematopoietic cells. We found low but detectable expression of CaMKI (CaMKIα) in all of the different hematopoietic cell types tested (FIG. 1C, first row). CaMKII consists of four distinct but highly homologous genes (α, β, γ, δ), and all of the hematopoietic cells displayed readily detectable levels of the CaMKIIγ isoform, while CaMKIIα expression was evident only in the immature, multipotent EML hematopoietic cells (Tsai, S. et al., Genes and Development 8:2831-2842 (1994)) and in normal immature bone marrow precursors (FIG. 1C, columns 2 and 8). In contrast with CaMKI and II, CaMKIV, while expressed in brain extracts (column 1), in the Jurkat T lymphoid cell line (not shown) and to a slight degree in MPRO cells (column 3), was not readily detected in any other hematopoietic cells. These Western blot observations were confirmed utilizing RT-PCR with RNAs from these different cell types which also indicated that CaMKIα and CaMKIIγ were the predominant CaMK mRNAs expressed by the NB4 and HL60 cell lines (not shown). We also quantitated both autonomous (Ca⁺⁺/CaM independent) and total (Ca⁺⁺/CaM dependent) CaMK enzyme activity in HL60 cell lysates following immunoprecipitation with different CaMK antibodies. Confirming the Western blots we observed that the highest level of both autonomous and total CaM kinase activity was observed in the CaMKIIγ immunoprecipitates (FIG. 1D, column 5). Lower enzyme activity was detected in CaMKI immunoprecipitates (FIG. 1D, column 2) while minimal enzyme activity is observed in CaMKIIα, β, or δ or CaMKIV immunoprecipitates (FIG. 1D).

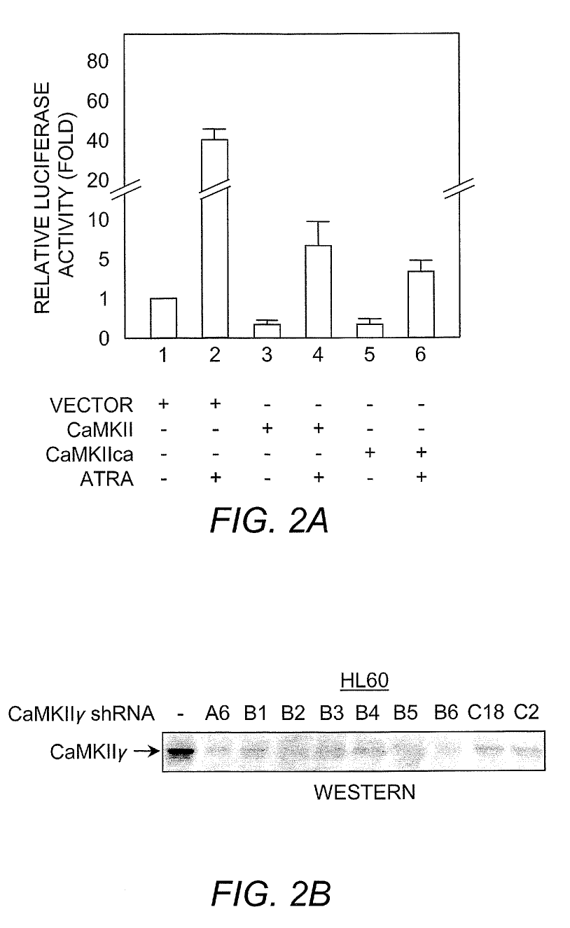

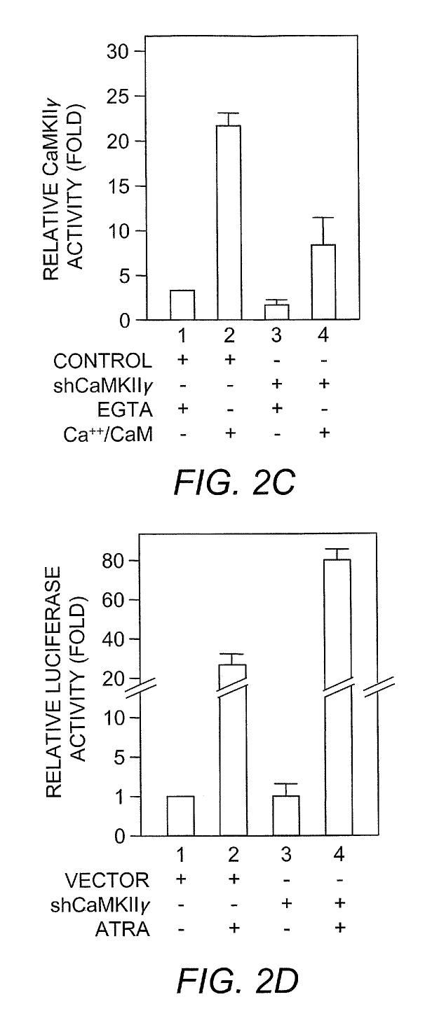

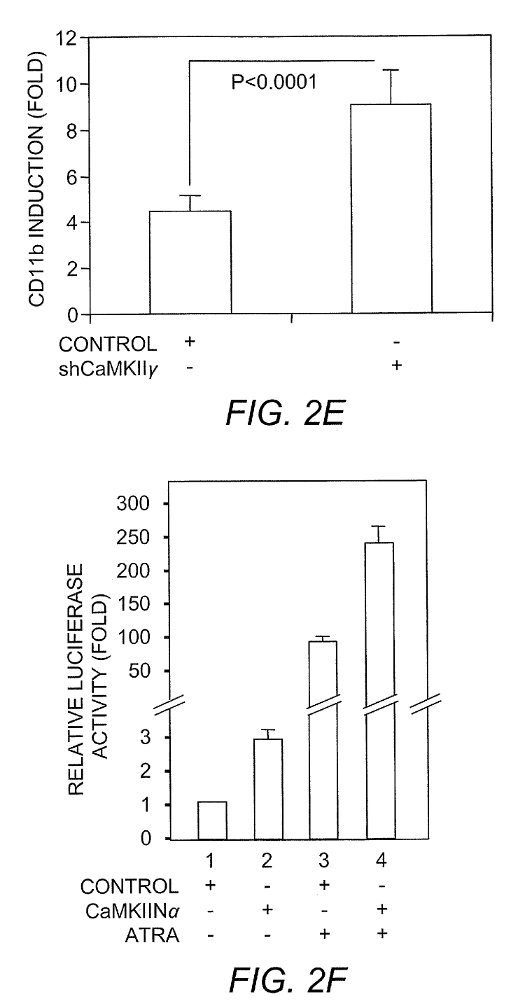

CaMKIIγ inhibits RAR activity in myeloid cells. The above studies indicate that CaMKIIγ is the CaM kinase predominantly expressed in myeloid cells, and we wished to determine whether this enzyme had any effect on RAR transcriptional activity. We utilized an expression vector harboring the full length CaMKII as well as one harboring a truncated CaMKII which lacks the calmodulin-binding domain and displays constitutive activity (ca) (Planas-Silva, M.D., and Means, A. R., Embo J 11:507-517 (1992)). Since we observe that the CaMK inhibitor, KN62 enhances RAR transcriptional activity (FIG. 1B), we expected that the co-transfected CaMKII might inhibit RAR activity. Indeed this is the case as we observed that both the full length and constitutively active (ca) CaMKII expression vectors significantly reduced RAR activity in the HL60 mycloid cells (FIG. 2A, compare lanes 3-6 with lanes 1,2). Similar results were also noted in transfected MPRO and NB4 myeloid cells (not shown). We also determined the effect of siRNA-mediated downregulation of CaMKIIγ on RAR activity in HL60 cells. We constructed retroviral vectors expressing CaMKIIγ shRNAs, stably transduced HL60 cells with these vectors and isolated six subclones (A6, B2, B5, B6, C2, C18) which exhibit diminished protein expression of CaMKIIγ compared with controls (FIG. 2B). These subclones also exhibited diminished CaMKIIγ enzymatic activity compared with control (empty) vector transduced HL60 cells (FIG. 2C). The CaMKIIγ shRNA transduced HL60 cells displaying this reduced CaMIIIγ expression/activity exhibit enhanced ATRA-induced RAR transcriptional activity compared with the empty vector transduced cells (FIG. 2D). Moreover, the HL60 subclones exhibiting this reduced CaMKIIγ expression/activity display an enhanced differentiative response to RA, which is reflected in their enhanced RA-induced expression of Cd11b, a surface antigen present on mature neutrophils (Kishimoto, T. K. et al., Science 245:1238-1241 (1989)) (FIG. 2E). Consistent with these observations we observed that co-transfection of the RARE reporter with an expression vector harboring CaMKIINα, a naturally occurring protein that specifically inhibits CaMKII activity (Chang, B. H. et al., Neuroscience 102:767-777 (2001)) enhances RARE reporter activity in HL60 (FIG. 2F). Taken together these observations indicate that CaMKIIγ inhibits RAR transcriptional activity in myeloid cells.

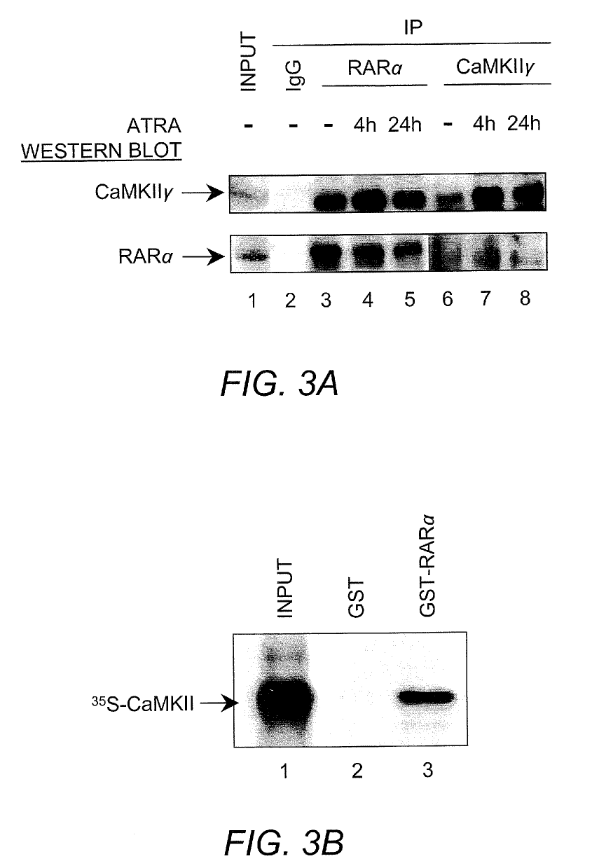

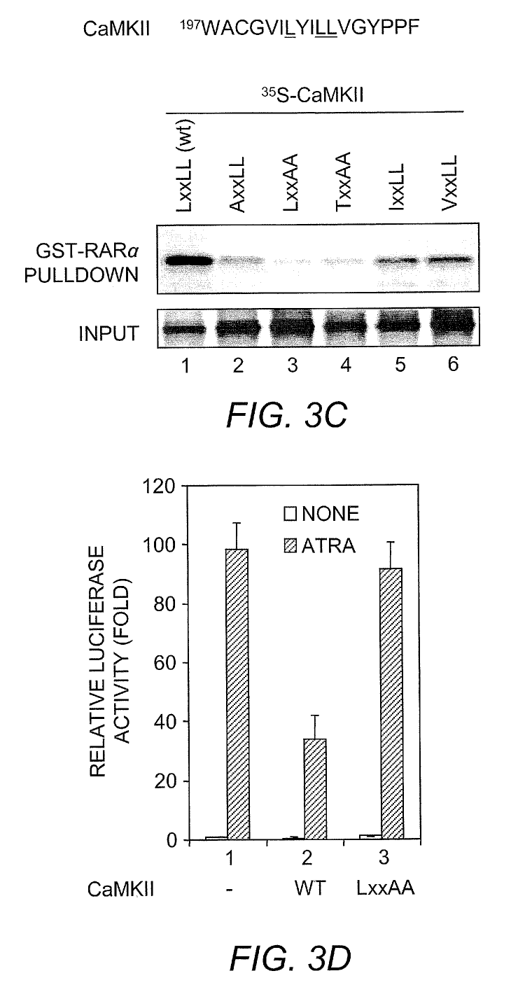

Physical interaction between CaMKIIγ and RARα is mediated through a CaMKII LxxLL motif. Since the above observations indicate that CaMKIIγ regulates (inhibits) RAR transcriptional activity, we performed coimmunoprecipitation (co-IP) studies to determine whether there was any physical interaction between CaMKIIγ and RARα in myeloid cells. We observed that immunoprecipitating endogenous RARα from both untreated and ATRA-treated HL-60 cells coimmunoprecipitates CaMKIIγ (FIG. 3A, lanes 3-5) and that antibodies to CaMKIIγ will co-IP RARα (FIG. 3A, lanes 6-8). These co-IP studies indicate that either there is a direct physical interaction between RARα and CaMKIIγ, and/or they are common members of a larger multiprotein complex. To determine whether CaMKII can directly bind RARα we assessed the CaMKII/RARα interaction utilizing in vitro GST pulldowns. A GST-RARα fusion protein bound CaMKII while GST alone did not (FIG. 3B). Although a potential “bridge factor” in the reticulocyte lysate might encourage this RARα/CaMKII interaction, nevertheless these observations suggest that RARα and CaMKII can directly bind each other.

Following ligand binding a conformational change occurs in the RA receptors promoting their interaction with a variety of different transcriptional co-regulators (coactivators). These coactivators harbor a signature LxxLL motif that directly mediates their interaction with RAR (7, 8). Inspection of the amino acid sequence of CaMKII indicates that the CaMKII α, β, γ, and δ isoforms all harbor a single LxxLL motif (LyiLL) within the kinase domain (FIG. 3C). To determine whether these LxxLL motif sequences are directly involved in mediating the interaction of CaMKII with RARα, we performed GST-RAR pulldowns utilizing in vitro translated CaMKII fusion proteins in which this LxxLL motif had been altered by site-directed mutagenesis. Mutating LxxLL motif residues within CaMKII results in significantly reduced binding to GST-RARα (FIG. 3C, compare lane 1 with lanes 2-6). Thus the CaMKII LxxLL motif appears important in mediating the binding of CaMKII to RARα.

To determine whether this LxxLL motif has any functional role in the CaMKII-mediated inhibition of RAR activity, we compared the activity of wild type vs. LxxLL motif-mutated CaMKII in regulating RAR reporter activity in transiently transfected HL60 myeloid cells. A CaMKII construct harboring a mutated LxxLL motif (LxxAA) that reduces binding to RAR (FIG. 3C, lanes 1 vs. 3) does not inhibit RAR activity compared with the parental (WT) CaMKII (FIG. 3D). Thus the CaMKII LxxLL motif appears important in regulating both the physical and functional interaction between CaMKII and RAR.

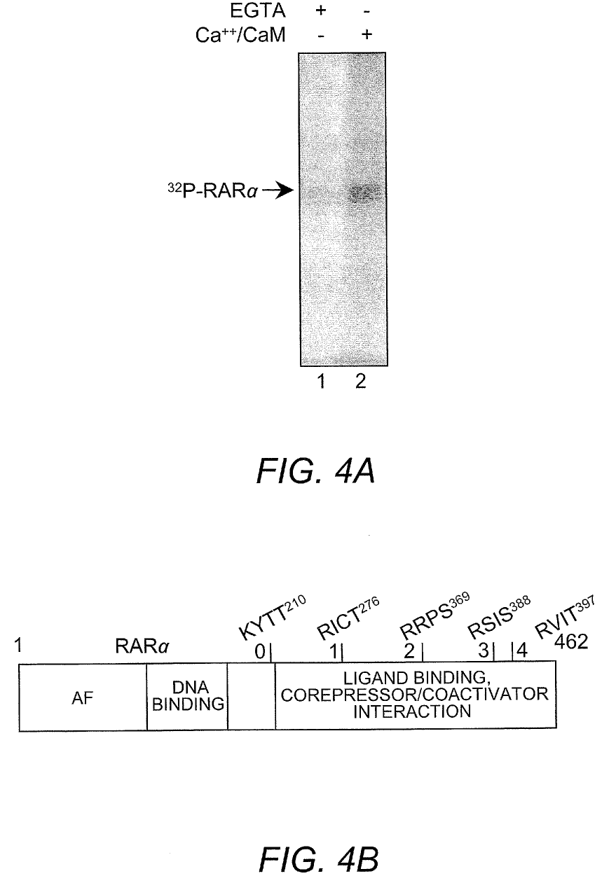

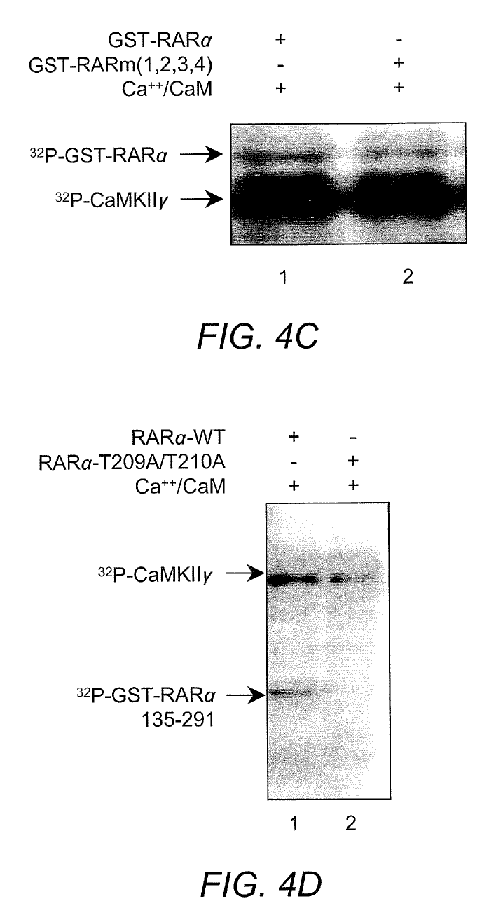

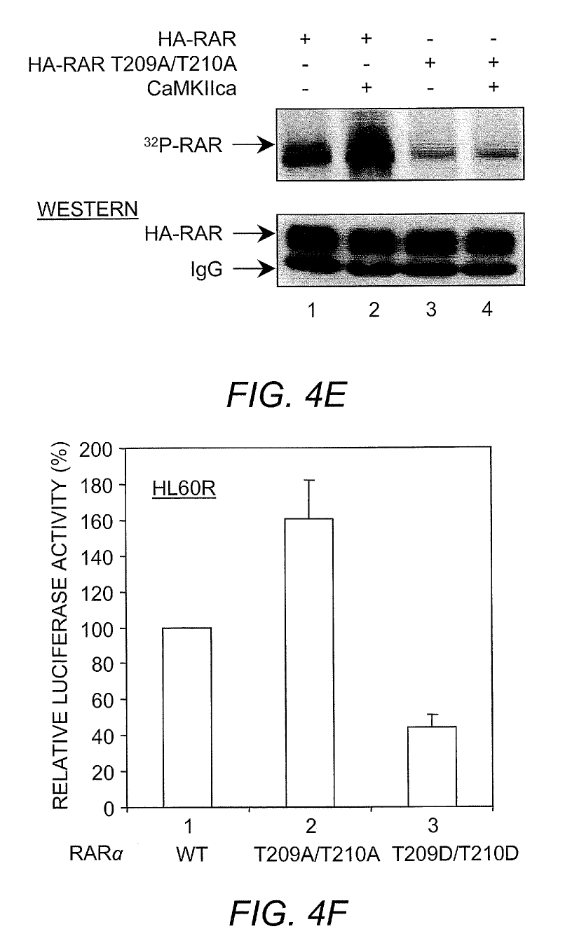

CaMKIIγ directly phosphorylates RARα. The direct binding of CaMKIIγ to RARα (FIG. 3) together with the CaMKII-mediated regulation of RAR transcriptional activity (FIG. 2) suggest that the RARs may be direct enzymatic substrates of the CaMKs. We performed in vitro kinase reactions and observed that CaMKII phosphorylates RARα in a Ca⁺⁺/CaM-dependent manner (FIG. 4A). A consensus substrate sequence targeted by the different CaMKs is R-x-x-S/T (Kishimoto, T. K. et al., Science 245:1238-1241 (1989)), and RARα harbors four such candidate CaMK target sequences within the C-terminal ligand binding/coactivator interaction domain. (These RARα sites are arbitrarily labeled 1-4 in FIG. 4B). To determine whether any of these four predicted substrate sites were phosphorylated by CaMKIIγ, we compared the CaMKII-mediated phosphorylation of GST-RARα fusion proteins in which all four of these potential phosphorylation sites were mutated to alanine (designated Gst-RARm(1,2,3,4). None of these four consensus sites in RARα appeared to be phosphorylated by CaMKIIγ, since this enzyme phosphorylates both the wild type and the four consensus site-mutated Gst-RARα to an equal extent (FIG. 4C, compare lanes 1 and 2). We identified another consensus CAMK site at T210 in the more N-terminal “hinge” region of RARα (arbitrarily designated site 0 (FIG. 4B)) that harbors a lysine rather than an arginine (KYTT210) (Stokoe, D. et al., Biochem J 296 (Pt 3):843-849 (1993)). We found that immunoprecipitated CaMKIIγ readily phosphorylated a GST-RARα fusion protein harboring amino acids 135-291 of RARα but not the same fragment harboring a TT→AA mutation at this RARα site O (FIG. 4D) indicating that the RARα site O is indeed a CaMKIIγ phosphorylation site.

To determine whether this CaMKIIγ phosphorylation site within RARα is also phosphorylated in vivo, we co-transfected ³²P labeled 293 cells with constitutively activated (ca) CaMKII together with wild type RARα vs the CaMKII site O—mutated RARα. The transfected CaMKII enhances the phosphorylation of the wild type RARα (FIG. 4E, compare lanes 1,2) but not the RARα that is mutated at the T209,T210 site (FIG. 4E, compare lanes 3,4). Thus the RARα CaMKII site that is phosphorylated in vitro (T209,210) is also phosphorylated in vivo.

To determine if CaMKIIγ-mediated phosphorylation of RARα at the T209,T210 site alters RARα activity, we compared the transcriptional activity of wild type RARα vs RARα harboring mutations at this site. We performed these studies in HL60R cells, which harbor a mutated RARα resulting in reduced endogenous RAR activity (Robertson, K. et al., Blood 80:1885-1889 (1992)). Compared with wild type RARα we observe that an RARα construct harboring nonphosphorylatable residues at the CaMKII site (T209A, T210A) displays enhanced RA-induced activity, while the RARα harboring phosphorylation-mimicking mutations at this same site (T209D, T210D) exhibits reduced transcriptional activity (FIG. 4F). Thus CaMKIIγ phosphorylation of RARα at the (T209, T210) site inhibits RARα transcriptional activity.

In summary we observe that CaMKIIγ directly phosphorylates RARα at a site within the hinge region of RARα (T209, T210), and this phosphorylation results in a downregulation of RARα transcriptional activity.

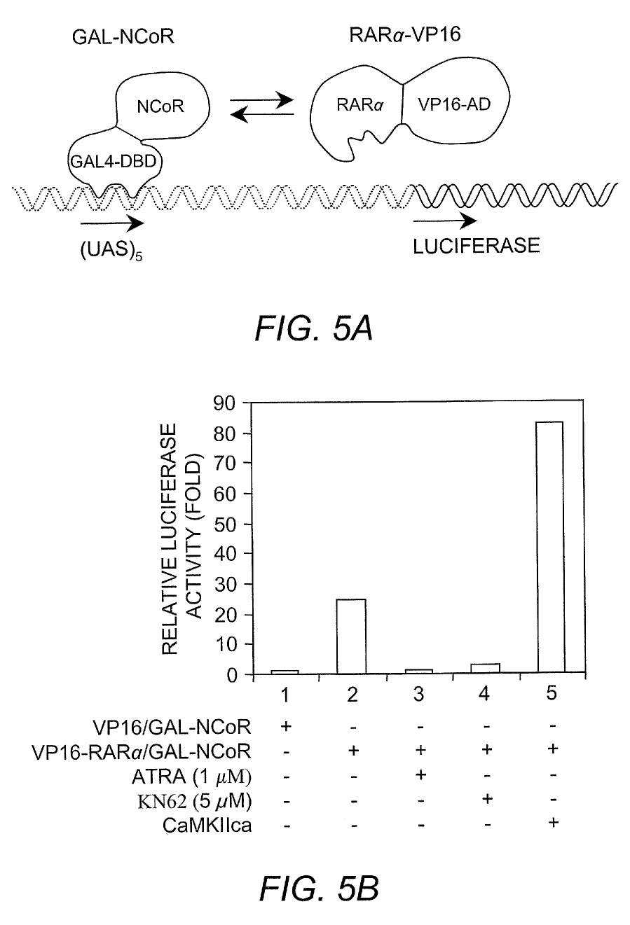

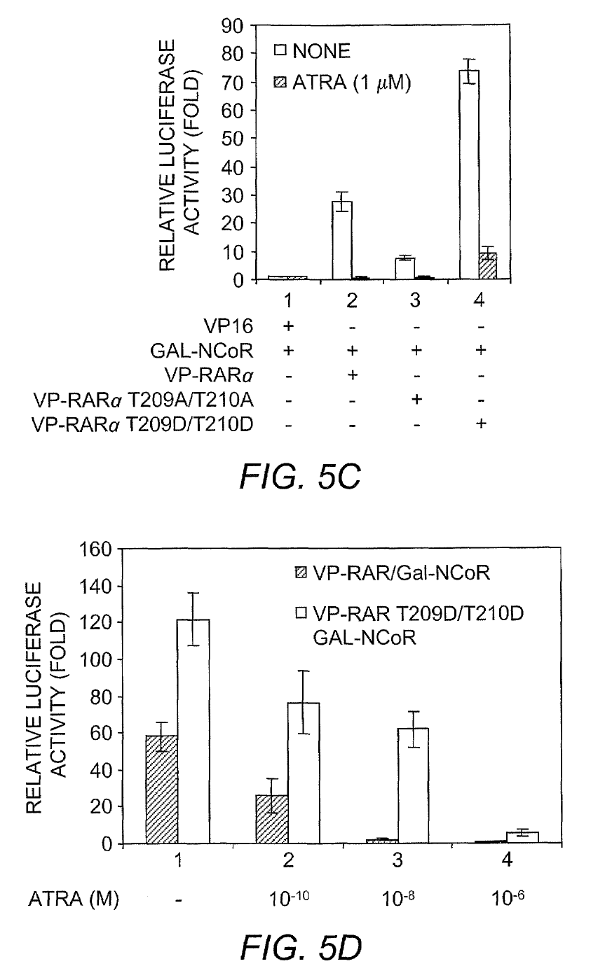

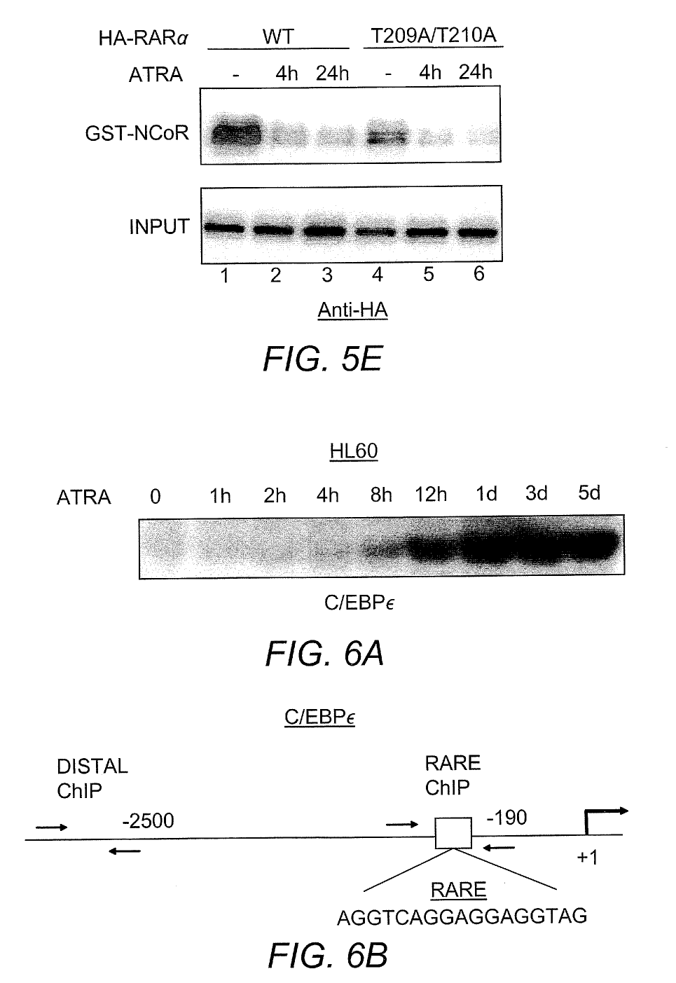

CaMKIIγ phosphorylation of RARα enhances RARα interaction with transcriptional corepressors. In the absence of RA the RARs physically interact with the corepressors, N-CoR/SMRT, and exposure to RA triggers corepressor release and coactivator recruitment leading to RAR transcriptional activation (Kao, H. Y. et al., J Biol Chem 278:7366-7373 (2003)). We utilized a mammalian two hybrid approach to determine whether the CaMKIIγ phosphorylation of RARα altered its interaction with corepressors/coactivators. Utilizing constructs designed to assess the RARα/N-CoR interaction (FIG. 5A) we observed that ATRA addition results in a marked decrease in the (UAS)₅-driven luciferase reporter activity (FIG. 5B, compare lanes 2 and 3) reflecting the previously observed ATRA-induced decrease in the RARα/N-CoR interaction (Kao, H. Y. et al., J Biol Chem 278:7366-7373 (2003)). Importantly, treatment of the transfected cells with KN62 also significantly inhibits luciferase activity (FIG. 5B, compare lanes 2 and 4) suggesting that the RARα/N-CoR interaction is also regulated by the CaMKs. Indeed co-transfection of a CaMKII expression vector enhances the luciferase reporter activity (FIG. 5B, compare lanes 2 and 5) suggesting that CaMKII phosphorylation of RARα enhances the RARα/N-CoR interaction. To directly test this hypothesis we mutated the RARα CaMKII site (T209,T210) and observed that the (T209A,T210A) mutated VP16-RARα fusion protein is associated with significantly reduced luciferase activity in the transfected cells compared with wild type (FIG. 5C, compare lanes 2 and 3). In contrast a phosphorylation-mimicking VP16-RAR T209D, T210D mutant leads to enhanced luciferase activity (FIG. 5C, compare lanes 2 and 4). Moreover, compared with the wild type VP16-RARα, this latter mutant requires significantly higher concentrations of ATRA to diminish the N-CoR/RARα interaction (FIG. 5D). Consistent with the mammalian two hybrid results we note that utilizing lysates from cells stably transduced with RARα vs the CaMKII site—mutated RARα, a Gst-N-CoR fusion protein interacts with the wild type RARα more efficiently than with the CaMKII site-mutated RARα (FIG. 5E, compare lanes 1 and 4). Taken together these observations indicate that CaMKIIγ phosphorylation of the RARα T209,T210 site enhances the RARα/N-CoR interaction.

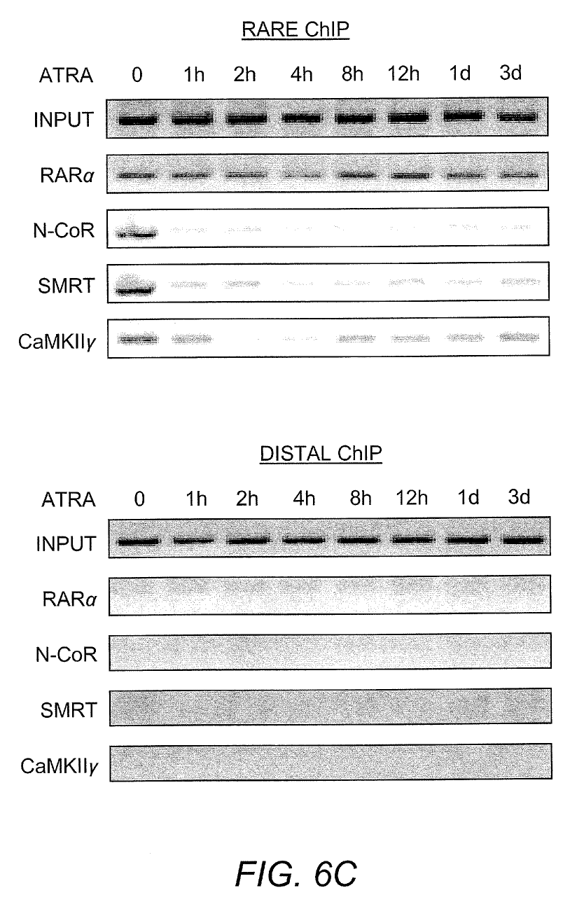

Association of CaMKIIγ with retinoic acid responsive myeloid gene promoters. The above observations indicate a potentially important role for CaMKIIγ in directly phosphorylating RARα and regulating its transcriptional activity in myeloid cells. We utilized chromatin immunoprecipitation (CHIP) to determine whether CaMKIIγ directly associated with the promoters of RAR regulated target genes. For these studies we chose to analyze the C/EBPε gene in HL60 cells since enhanced C/EBPε expression occurs during RA-induced differentiation of HL60 (FIG. 6A), and this is mediated through a specific RARE located 190 bp upstream of the C/EBPε translation start site (Park, D. J. et al., J Clin Invest 103:1399-1408 (1999)). For these ChIPs PCR primers flanking the C/EBPε RARE were utilized together with control primers flanking a distal C/EBPε genomic region approximately 2500 bp upstream (FIG. 6B). Both before and after RA-induction we observed a consistent association of RARα with the C/EBPε RARE but not with the upstream distal region (FIG. 6C). Following RA treatment there was the expected rapid displacement from this RARE of the RAR corepressors, N-CoR and SMRT (FIG. 6C). We observed that CaMKIIγ was also associated with this RARE in uninduced cells compared with the upstream control region. Interestingly RA treatment was accompanied by dissociation of CaMKIIγ from this RARE, and these changes were observed as early as 1-2 hours following RA exposure (FIG. 6C).

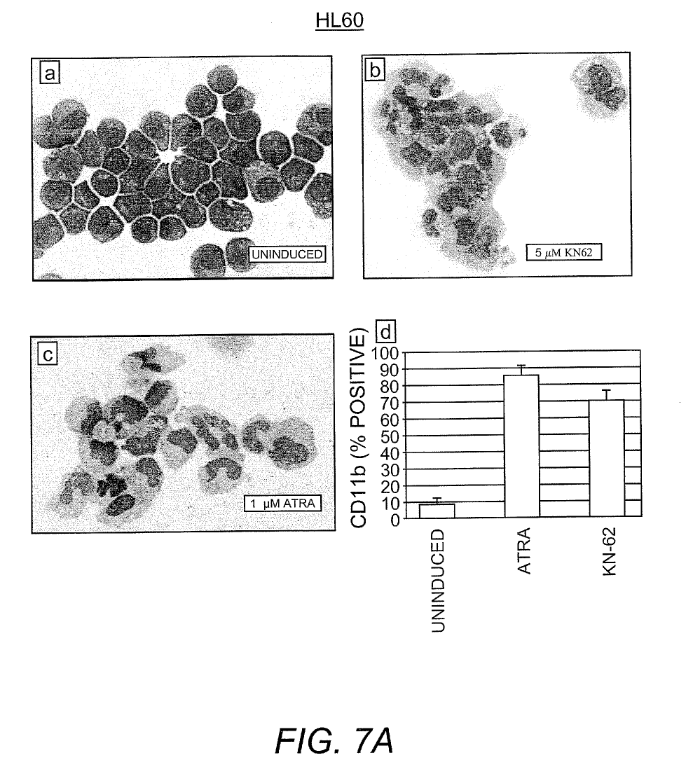

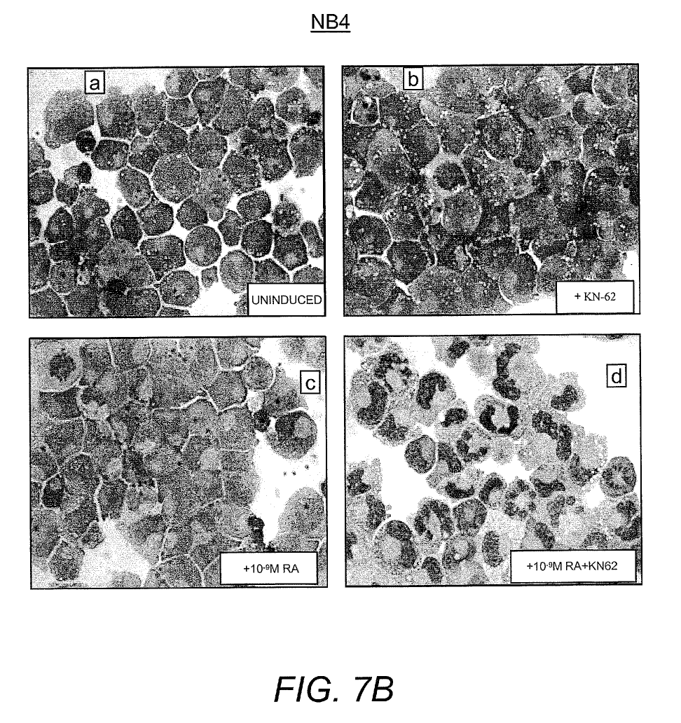

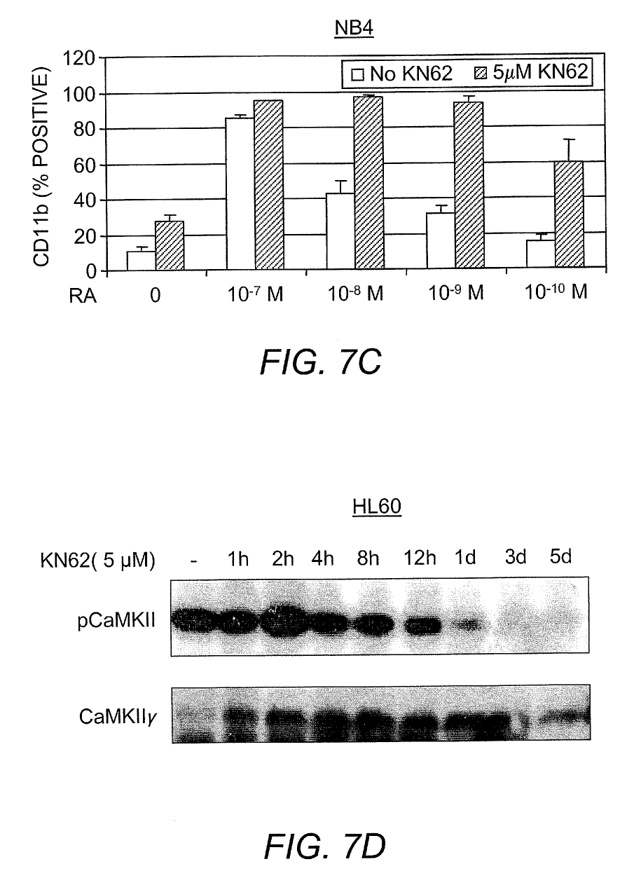

The CaM kinase inhibitor KN-62 is a potent inducer of myeloid cell differentiation. The RA-induced myeloid differentiation of HL60 cells is mediated directly through RARα (Fong, Y. L. et al., J Biol Chem 264:16759-16763 (1989). Our observation that KN62 enhances RAR transcriptional activity in HL60 cells (FIG. 1B) suggests that this compound might also regulate the differentiation of these cells. We observed that KN-62 as a single agent is a potent inducer of HL-60 differentiation enhancing both morphologic differentiation as well as expression of the neutrophil antigen, Cd11b, (FIG. 7A). Similarly as a single agent KN-62 enhanced the granulocytic differentiation of the MPRO cell line (not shown). In contrast with the HL-60 and MPRO cells, we observed in the NB4 promyclocytic leukemia cell line, which harbors the PML-RARα fusion gene (Lanotte, M. et al., Blood 77:1080-1086 (1991)), that KN-62 alone is a relatively poor inducer of morphologic granulocytic differentiation (FIG. 7B, compare panels a and b). However, KN-62 in combination with relatively low concentrations of RA (10⁻⁹M-10⁻¹⁰M) induces a marked increase in the granulocytic differentiation of NB4 cells as assessed by morphologic differentiation (FIG. 7B, compare panels c and d) as well as the induction of Cd11b surface antigen expression (FIG. 7C). Thus the CaMK inhibitor KN-62 not only enhances RAR transcriptional activity but either as a single agent or in combination with relatively low doses of RA can induce/enhance the differentiation of different myeloid leukemia cell lines.

KN62 can inhibit multiple different CaMKs by interfering with CaMK binding to Ca⁺⁺/CaM. To determine whether KN62 might alter CaMKII activation in differentiating myeloid cells, we utilized phospho-specific CaMKII antibodies that identify the activated CaMKII. Autophosphorylation of threonine-286/287 (CaMKIIα: T286, CaMKIIβ,γ,δ: T287) of CaMKII is associated with autonomous enzyme activation (Fong, Y. L. et al., J Biol Chem 264:16759-16763 (1989), and this autophosphorylated CaMKII can be detected utilizing a CaMKII T286/7 phospho-specific antibody. In HL60 cells KN62 induced a marked reduction in the activated, autophosphorylated CaMKIIγ that was readily apparent within a day following KN62 exposure (FIG. 7D). In contrast KN62 did not induce any significant changes in the expression of total CaMKIIγ in these differentiating cells (FIG. 7D). In NB4 cells KN62 alone had little effect on the levels of autophosphorylated CaMKIIγ, but the combination of KN62 and relatively low concentrations of RA, which induces NB4 differentiation (FIG. 7B,C), also induces a reduction in the autophosphorylated CaMKIIγ (FIG. 7E). Thus the KN62 mediated enhanced differentiation of HL60 and NB4 cells is associated with reduced amounts of the activated, autophosphorylated CaMKIIγ.

### Discussion

CaMKIIγ is a member of the RAR transcription complex. Our studies have revealed a novel, previously unexplored cross talk between Ca⁺⁺-regulated and RAR signal transduction pathways. Indeed we observe that CaMKII, which is one of the most widely studied Ca⁺⁺ regulated enzymes and which is a critical regulator of neuronal cell development and activity, is an important regulator of RAR transcriptional activity in myeloid cells. Indeed a number of our observations indicate that CaMKIIγ is a direct functional component of the RAR transcription complex. First, we observe in transient transfection assays that CaMKII inhibits the transcriptional activity of the RA receptors (FIG. 2A). Moreover, CaMKII directly binds to the RARs through a CaMKII signature LxxLL motif (FIG. 3C), a motif also present in NR transcriptional coactivators (8). Finally our ChIP studies indicate that CaMKIIγ localizes in vivo to RA response elements (RAREs) within myeloid target gene promoters (FIG. 6). Notably RA treatment triggers a reduced association of CaMKIIγ, which inhibits RAR activity, with this RARE (FIG. 6C). This RA-induced reduction in CaMKIIγ association with the RARE may serve to relieve the inhibitory effect of CaMKIIγ on RAR activity and may be an important mechanism to regulate/amplify RAR activity following RA induction. Interestingly some CaMKIIγ appears to return to the promoter 4-8 hours after ligand addition (FIG. 6C), which may be indicative of the cyclical interaction of corepressors/coactivators previously observed at the gene targets of other nuclear hormone receptors (Metivier, R. et al., Cell 115:751-763 (2003)).

CaMKIIγ phosphorylation of RARα enhances its interaction with transcriptional co-repressors. The multicomponent RAR transcription complex may consist of at least 30-40 or more proteins, and multiple substrates may be involved in the CaMKII-mediated downregulation of RAR activity. Nevertheless, our experimental observations indicate that the RARs themselves appear to be a critical substrate of CaMKIIγ. Indeed we have identified a target site within RARα (T209,T210) which is directly phosphorylated by CaMKIIγ both in vitro and in vivo. This site is evolutionarily conserved in all of the different RARα, β, and γ isoforms from multiple different species and lies within the “linker” region of RAR that separates the DNA binding and AF-2 domains of RAR. We observe that CaMKIIγ-mediated phosphorylation at RARα T209,T210 enhances the RARα interaction with the N-CoR transcriptional corepressor (FIG. 5) which likely leads to reduced RAR transcriptional activity. This CaMKII site likely does not make direct contact with N-CoR since corepressor interaction with RARα appears to map more C-terminal to this site (Perissi, V. et al., Genes Dev 13:3198-3208 (1999); Nagy, L. et al., Genes Dev 13:3209-3216 (1999)). Rather phosphorylation of this CaMKII site within the RARα linker region likely results in a steric change in RARα that enhances its interaction with N-CoR. The direct binding of N-CoR to the RA receptor is a critical mediator of RAR transcriptional repression since N-CoR acts as a scaffold protein to recruit different transcriptional repressors including the HDACs to RA responsive promoters. Thus this CaMKIIγ-mediated enhancement of the RARα/N-CoR interaction likely explains the CaMKIIγ-mediated inhibition of RARα transcriptional activity.

Pharmacological CaMK inhibitors trigger myeloid leukemia cell differentiation. Particularly compelling evidence for the physiological importance of the CaMKII—mediated regulation of RAR activity is our experimental observation that KN-62, a pharmacological inhibitor of the CaMKs, enhances both RAR transcriptional activity (FIG. 1B) and the differentiation of certain myeloid leukemia cell lines (FIG. 7). Acute myelogenous leukemia (AML) is a heterogenous disease, and successful therapy with RA has been generally confined to those leukemias harboring RARα chromosome translocations. Indeed our preliminary studies indicate that KN-62 does not induce the differentiation of myeloid leukemia cell lines such as K-562, KCL-22 and Kasumi that do not harbor such RARα chromosome translocations and are generally unresponsive to RA (not shown). Moreover, in contrast with wild type HL60, we do not observe any KN62-mediated differentiation of HL60R cells, which harbor a mutated RARα or of NB4 subclones that are unresponsive to ATRA. These initial observations suggest that any therapeutic application of KN-62 may be limited to those myeloid leukemias that display at least some sensitivity to the differentiative effects of RA.

How does KN62 trigger the differentiation of certain myeloid leukemia cells? Since KN-62 inhibits the binding of Ca⁺⁺/CaM to the CaMKs, this compound can potentially inhibit the multiple different CaMKs which are all activated by Ca⁺⁺/CaM (Means, A. R., Mol Endocrinol 14:4-13 (2000)). However, our data strongly suggest that CaMKIIγ is a critical KN62 target for HL60 differentiation since CaMKIIγ is the predominant CaMK expressed in myeloid cell lines (FIG. 1C, D), CaMKII inhibits RAR transcriptional activity (FIG. 2A), and RNAi-mediated inhibition of CaMKIIγ enhances both RA-induced RAR activity and RA-induced myeloid differentiation (FIG. 2D, E). Moreover, in HL60 cells, which harbor a constitutively activated (autophosphorylated) CaMKIIγ, KN62 inhibits this CaMKIIγ autophosphorylation (FIG. 7D). Indeed our studies would suggest that pharmacological or peptide inhibitors that are selective for CaMKIIγ may be of particular value in the therapy of certain myeloid leukemia cells.

### Methods

Antibodies and chemical reagents. Antibodies against RARα, βtubulin, CaMKIIα, CaMKIIβ, CaMKIIγ, CaMKIIδ, CaMKIα, as well as phosphospecific antibodies against CaMKII were all purchased from Santa Cruz Biotechnology. Antibodies against N-CoR and SMRT were from Upstate Biotechnology. CaMKIV antibody was from BD Biosciences. Anti-FLAG antibody was from Sigma while anti-HA antibody from Novachem. Quantitation of CD11b surface antigen expression was performed utilizing the fluorescent-activated cell sorter (FACS) with PE-conjugated anti-CD11b antibody from Becton Dickinson. KN-62, KN-93 and ionomycin were obtained from Calbiochem. ATRA and calmodulin agarose beads were from Sigma.

Protein extracts, Western blotting and co-immunoprecipitations. Westerns and co-immunoprecipitations were performed as previously detailed (Si, J., and Collins, S. J., Blood 100:4401-4409 (2002)).

CaM kinase assays. To assess the activity of immunoprecipitated CaMKs the immunoprecipitates were incubated for eight minutes at 30° in 40 μl of a buffer containing 50 mM HEPES, ph 7.5, 10 mM Magnesium acetate, 20 mM β glycerol phosphate, 0.5 mM DTT, 50 μM cold ATP, 1 μM PKA inhibitor (Sigma), 1 μM PKC inhibitor (Sigma) and 10 μCi of [γ-³²P]ATP (3000 Ci/mmol) (Perkin Elmer, Life and Analytical Sciences). For assessing Ca⁺⁺/calmodulin independent activity the above buffer contained 1 mM EGTA. For assessing Ca⁺⁺/calmodulin dependent activity the buffer contained 1 mM CaCl₂, 1 μM calmodulin (Calbiochem) without EGTA. The peptide substrate included autocamtide-2 (KKALRRQET*VDAL) (Hanson, P. I. et al., Neuron 3:59-70 (1989)) to assess CaMKII activity, syntide-2 (PLARTLS*VAGLPGKK) (Hashimoto, Y. et al., Arch Biochem Biophys 252:418-425 (1987)) to assess CaMKI and the aa345-358 fragment of CaMKII-6 (KSDGGVKRKSS*SS) to assess CaMKIV activity. Following incubation the phosphorylated substrate was separated from the residual [γ-³²P]ATP using P81 phosphocellulose paper and quantitated with a scintillation counter. For the in vitro kinase assays either in vitro translated or immunoprecipitated CaMKs were incubated with bacterially purified GST-RARα fusion proteins attached to glutathione beads. The beads were washed, boiled and then subjected to SDS-PAGE.

ChIP. HL60 cells incubated for various periods of time after ATRA (1 μM) treatment were harvested and crosslinked using 1% formaldehyde for 15 minutes at 37° and incubated another 15 minutes at room temperature with 125 mM glycine. Cells then were rinsed and washed three times with cold PBS. The cell pellets were resuspended in 0.2-2 ml of SDS lysis buffer (1% SDS, 10 mM EDTA, 5 0 nM Tris-HCL, pH 8.1) with protease inhibitors(1 μg/ml leupeptin, 1 g/ml aprotinin, 1 mM PMSF, 1 μg/ml pepstatin A, 10 μM antipain, 10 μM bestatin, 1 mM NaF, and 1× phosphatase inhibitor cocktail I/II) and sonicated three times at 20 s each to reduce DNA length to 200-800 bp. Supernatants were collected by centrifugation for 10 minutes at 13,200 rpm at 4° and diluted in 10 fold dilution buffer (0.01% SDS, 1.1% Triton X-100, 1.2 mm EDTA, 16.7 mM Tris-HCl, pH 8.1, 167 mM NaCl) with the above protease inhibitors. The diluted cell extracts (500-1000 μg) were incubated with 4 μg or 10 μl antibodies overnight at 4° followed by 50 ul of Salmon Sperm DNA/protein A/G-Sepharose beads for 4 hr. Pellet beads were washed sequentially for 5 minutes each in Buffer A (0.1% SDS, 1% Triton X-100, 2 mM EDTA, 20 mM Tris-HCl, pH8.1, 150 mM NaCl), Buffer B(0.1% SDS, 1% Triton X-100, 2 mM EDTA, 20 mM Tris-HCl, pH8.1, 500 mM NaCl), Buffer C(250 mM LiCl, 1% NP40, 1% deoxyxholate, 1 mM EDTA, 10 mM Tris-HCl, pH8.1) and TE buffer. Immune complexes were eluted with 1% SDS, 0.1M NaHCO₃ and incubated at 65° overnight to reverse the crosslinking. DNA fragments were isolated with QLAquick spin column (Qiagen). PCR was performed for 20-30 cycles. PCR primers flanking the C/EBPε promoter RARE utilized for the “RARE ChIP” include 5′ AAGAGAGGCATTGACTAC 3′ (forward) and 5′ACCTGCTCTTGAGGCACCCCTT 3′(reverse). Primers utilized for the upstream control region “distal ChIP” include 5′CCTGACATCCAGAGCCCTGCCT 3′ (forward) and 5′ AGTAGCTTCAGTGAACTGGGCAC 3′ (reverse).

CaMK RNAi constructs. Separate cassettes harboring the U6 (−332 to +1) or H1 (−345 to +1) promoters were inserted into the EcoR1-BaMH1 cloning site of the LXSN retroviral vector (M28248). These cassettes harbor sites into which synthetic oligos corresponding to sense and antisense strands of human CaMKIIγ separated by a 9 bp spacer harboring an XhoI site (5′TTCTCGAGA-3′) were cloned. Three different sequences from the human CaMKIIγ coding region (NM_{—}172172) were chosen (453-473; 618-639; and 790-812) and arbitrarily labeled “A, B, C”. These sequences were cloned into the above vectors, which were then transfected into the PT67 packaging cell line, and the retroviral particles were utilized to infect HL-60 cells as previously detailed (36). G418 resistant HL60 clones were isolated and screened by Westerns to compare CaMKIIγ expression levels. Those HL60 subclones exhibiting reduced CaMKIIγ expression (i.e. A6, B2, B5, B6, C2, C18) were further analyzed for immunoprecipitable CaMKII activity, RAR transcriptional activity and differentiative response to RA.

Transient transfections and luciferase assays. Murine PT67 fibroblasts were transfected utilizing calcium phosphate precipitation. All hematopoietic cell lines were transiently transfected by electroporation as previously detailed (Johnson, B. et al., Molecular and Cellular Biology 19:3372-3382 (1999)). In the transient transfections the LXSN vector (M28248) served as the expression vector for the wild type and site-directed mutagenized CaMKII and RARα coding region cDNAs. Human cDNA homologous to the coding region of rat CaMKIINα, a CaMKII inhibitory protein, was obtained by RT-PCR from HL60 RNA and cloned into the LXSN vector. The RA-responsive luciferase reporter harbors the RARE from the RARβ2 promoter as previously detailed (Si, J., and Collins, S. J., Blood 100:4401-4409 (2002)). The PON838 plasmid, a β-galactosidase reporter driven by the β-actin promoter, was used as an internal control to determine transfection efficiency for calculating relative luciferase activity.

Mammalian two hybrid constructs. The luciferase reporter harboring five Gal4 binding sites was previously described (Johnson, B. et al., Molecular and Cellular Biology 19:3372-3382 (1999)). The complete coding sequence of the human RARα (NM_{—}000964) was amplified by PCR and cloned into the PSG-VP16 vector (Butler, A. J., and Parker, M. G., Nucleic Acids Res 23:4143-4150 (1995)). The N-CoR C-terminal receptor interacting domain (aa 1946-2453) was amplified by PCR and cloned into the EcoRI site of the GAL_{dbd} (1-147) expression vector, pSG424 (Sadowski, I., and Ptashne, M., Nucleic Acids Research 17:753-754 (1989)).

Site-directed mutagenesis on the different CaMK and RARα or VP16-RARα constructs were performed utilizing the “Quikchange” procedure (Stratagene).

RT-PCR. The specific primers utilized to assess the different CaMK mRNA levels as well as the RT-PCR conditions are available on request.

GST-pulldowns. GST-fusion proteins were purified from bacteria using glutathione-4B Sepharose (Amersham Biosciences). ³⁵S labeled proteins were translated in vitro using a T₇ Quick Coupled transcription/translation system (Promega). The products were incubated overnight at 4° with the GST-fusion proteins attached to the glutathione sepharose beads. The beads were then washed, boiled and subjected to SDS-PAGE.

### Example 2

### Activated Ca⁺⁺/Calmodulin Dependent Protein Kinase IIγ is a Critical Regulator of Myeloid Leukemia Cell Proliferation

In previous studies we have observed that CaMKIIγ is the CaMKII isoform that is preferentially expressed in myeloid cells, and this enzyme inhibits the retinoic acid induced differentiation of myeloid leukemia cell lines by phosphorylating and inhibiting the transcriptional activity of the retinoic acid receptor (RARα) (Si J, Mueller L, Collins S J., CaMKII regulates retinoic acid receptor transcriptional activity and the differentiation of myeloid leukemia cells. J Clin Invest 117: 1412-21 (2007)). In these previous efforts we observed that CaMKII inhibitors enhance the differentiation of certain myeloid leukemia cell lines, but this effect was only noted in leukemias that were responsive to retinoic acid In the present study we extend these studies beyond the retinoic acid responsive myeloid leukemias and describe a critical and central role of CaMKIIγ in regulating the proliferation of a wide variety of myeloid leukemia cells. We observe that autophosphorylated CaMKIIγ is commonly present in myeloid leukemias. Inhibitors of CaMKIIγ including pharmacological agents and dominant negative constructs inhibit myeloid leukemia cell proliferation, and this is associated with the inactivation of multiple signal transduction pathways including MAPK p44,42, Stat3/Stat5, and GSK 3β/β catenin. Moreover, we observe that Stat3 is directly phosphorylated by CaMKII at Ser727. These observations highlight the importance of CaMKII in regulating the proliferation of myeloid leukemia cells and indicate inhibiting CaMKIIγ activity in the targeted therapy of myeloid leukemia.

### Materials and Methods

Cell lines. All leukemia cell lines are cultured at 37° in RPMI supplemented with 5% heat-inactivated fetal calf serum.

Antibodies and Western blots. Protein extracts, immunoprecipitations and Western blots were performed as previously detailed (Si J, Collins S J. IL-3-induced enhancement of retinoic acid receptor activity is mediated through Stat5, which physically associates with retinoic acid receptors in an IL-3-dependent manner. Blood 100: 4401-9 (2002)). Antibodies against CaMKI, CaMKIIγ, CaMKIV, c-myc, Stat3, Stat5, calmodulin, as well as the phosphospecific CaMKII (T286, 287) and phosphospecific CaMKI (T177) antibodies were from Santa Cruz Biotechnology. Antibodies against phospho-Stat1 (Ser727), phospho-Stat3 (Ser727 and Tyr 705), phospho-Stat5 (Tyr 694), phospho-p44/42 MAP kinase (Thr202/Tyr 204), p44/42 MAP kinase, phospho-p38 MAP kinase (Thr 180/Tyr 182), phospho-GSK3β (Ser9) were from Cell Signaling Technology. Antibodies against Stat1, GSK3β, β-catenin, PP2Ac, and cyclin D1 were from BD Biosciences.

Chemicals. Different inhibitors of specific signal transduction pathways including AG490, Jak3 inhibitor II, PP1, PD98059, UO 126, SB 203580, wortmannin, LY 294002, JNK inhibitor II, raf kinase inhibitor I, pho kinase inhibitor, RO-31-8220, GÖ 6983, cucurbitacin I, AMP kinase inhibitor compound C, KN62, KN93 and STO609 were all from Calbiochem. All-trans retinoic acid (ATRA), phorbol myristate acetate (TPA), arsenic trioxide and SB216367 were from Sigma.

Patient AML and normal CD34+ samples. Patient AML samples were obtained from the Childrens Oncology Group (COG) bank of AML cells. Ficoll gradient centrifugation was utilized to isolate mononuclear cells from bone marrow aspirates from newly diagnosed patients with AML. These cells consisted of >80% blasts by morphology and were stored frozen under liquid nitrogen. Normal CD34+ cells were immunopurified from G-CSF mobilized, leukophoresed peripheral blood stem cell donors. Appropriate IRB approval was obtained for utilizing these patient and normal donor samples which are anonymized such that individuals cannot be identified.

GST-fusion proteins. The coding region of the human Stat3α gene was amplified by RT-PCR and subcloned into the GST fusion protein expression vector, pGEX5x.1. The GST-Stat3 fusion protein was purified from bacteria extracts using glutathione-4B Sepharose (Amersham Biosciences).

Expression vectors. A cDNA harboring the coding sequence of amino acids 1-290 of CaMKIIγ was cloned into the LXSN retroviral expression vector (M28248). Utilizing site-directed mutagenesis we mutated Lys⁴³ of this construct to methionine to generate a kinase deficient CaMKII mutant labeled kdCaMKII.

CaMKIIγ cDNA amplification and sequencing. Total cellular RNA from the leukemia cell lines and normal tissue was isolated by Trizol reagent (Invitrogen). First strand cDNA was made by the superscript amplification system (Invitrogen) with the oligo(dT)₁₂₋₁₈ primer. To determine the expression of the different CaMKIIγ isoforms we performed RT-PCR utilizing primers flanking the variable region of CaMKIIγ including:

| | |
|---|---|
| Sense | 5′ CGTCAGGAGACTGTGGAGTGT |
| Antisense | 5′ TCACTG CAG CGG TGC GGC AGG |

To amplify and sequence the complete CaMKIIγ coding sequence the cDNA from the HL60 and NB4 cell lines was amplified in separate reactions with two different primer pairs flanking the amino terminal and COOH terminal regions of the CaMKIIγ coding sequence. The sequence of these primers as well as the GAPDH control primers are available on request. The PCR products were isolated and subcloned into the TA cloning vector for DNA sequencing.

Transient transfections and luciferase assays. K562 cells were transiently transfected utilizing electroporation. 5×10⁶ cells were electroporated at 500 μF, 250 V, and cultured for 24 h under standard conditions. NIH3T3 cells were transiently transfected utilizing lipofectamine 2000 (Invitrogen). Stat3 transcriptional activity was determined in K562 cells transfected with the pSIE-tk-LUC luciferase reporter which harbors a Stat3 binding site (CCTTAATCCTTCTGGGAATTCTGGCT). The β-gal reporter driven by the β-actin promoter was used as an internal control for transfection efficiency.

CaMKIIγ RNAi constructs. Three LXSN based plasmids harboring three different synthetic human CaMKIIγ oligos designed to generate shRNA hairpins were constructed as previously detailed (Si, Mueller and Collins, supra (2007)). We synthesized and inserted an additional oligo generating an shRNA corresponding to human CaMIKIIγ (NM_{—}172171) coding region (954-976) into the pLL3.7 lentiviral vector plasmid which harbors a GFP marker (Rubinson D A, et al., Nat Genet. 33: 401-6 (2003)). These four plasmids were simultaneously electroporated into K562 cells which were selected in G418 (800 ug/ml) for 10 days. GFP+ cells from this culture were then FACS enriched and cell lysates subjected to Western blot analysis.

Construction and utilization of doxycycline inducible expression vectors. A full length CaMKIIγ cDNA was mutated at Lys⁴³ to methionine to render it kinase deficient. It was then FLAG-tagged at the amino terminus and cloned into the pTRE-Tight expression vector which harbors a tet-responsive element available from CLONETECH.). This plasmid was electroporated into K562 cells together with the Clontech pTet-On Advanced plasmid, which codes for a doxycycline-regulated transactivator protein. The electroporated cells were selected in G418 (800 ug/ml) for 10 days. Cell lysates for western blot analysis were obtained from these transfected cells as well as from the same cells treated for two days with doxycycline (1 ug/ml).

### Results

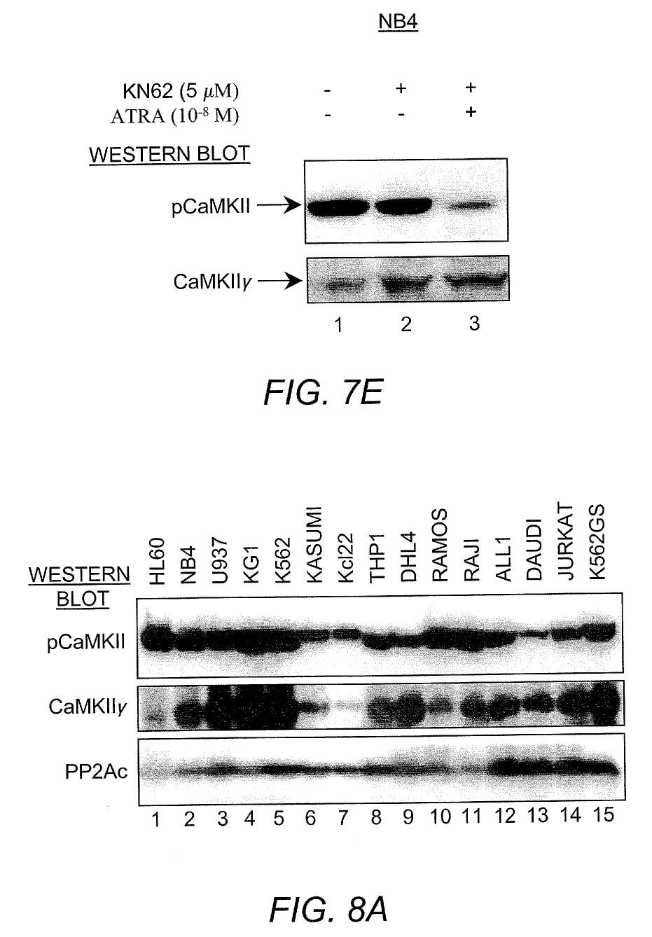

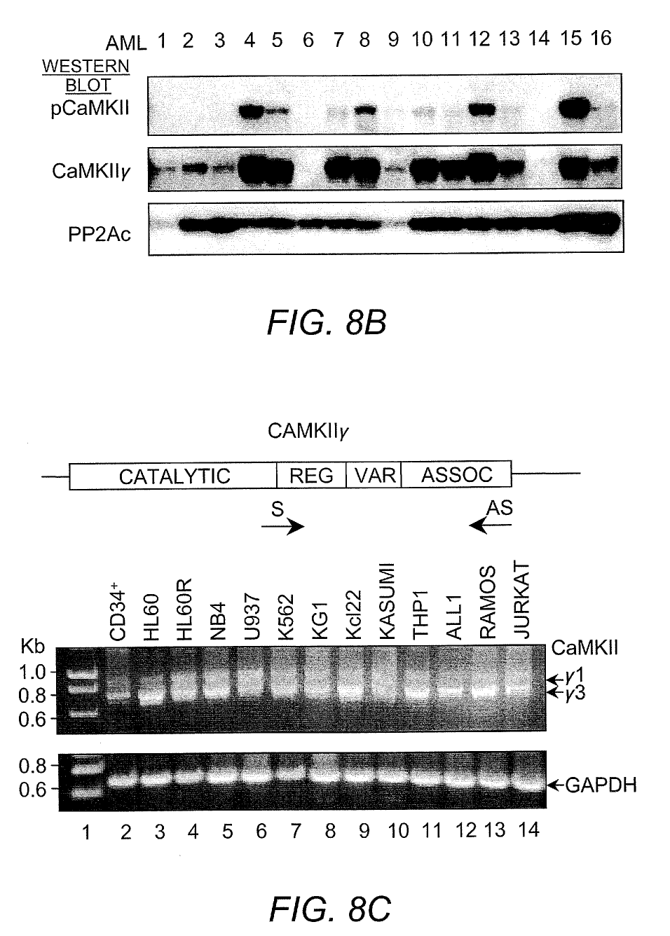

Activated CaMKIIγ is frequently present in myeloid leukemia cell lines and primary AML cells. We previously observed that CaMKIIγ is the CaMKII isoform that is predominantly expressed in myeloid cells. The binding of Ca⁺⁺/calmodulin to the inactive CaMKIIγ triggers autophosphorylation of this enzyme at threonine 287 leading to autonomous enzyme activity that is relatively independent of further Ca⁺⁺/calmodulin regulation (Fong Y L et al., J Biol Chem 264: 16759-63 (1989); Miller S G et al., Neuron 1: 593-604 (1988); Lou L L, et al., J Neurosci 9: 2020-32 (1989)). This autophosphorylated CaMKIIγ can be detected utilizing a CaMKII threonine 287 phospho-specific antibody. Utilizing this antibody on Western blots we observed that the autophosphorylated (activated) CaMKIIγ was invariably present in different human leukemia cell lines (FIG. 8A). To further investigate the role of CaMKIIγ in the pathogenesis of human leukemia the present study focuses on the myeloid leukemias, and we observe that the activated, autophosphorylated CaMKIIγ is also present at varying levels in the majority (11/16) of primary patient AML samples (FIG. 8B). In contrast Western blots did not reveal any detectable autophosphorylated CaMKIIγ in three different normal peripheral white blood cell samples (not shown).

Since alternative splicing within a variable region of CaMKIIγ generates a number of different isoforms (Tombes R M, et al., Gene 322: 17-31 (2003)), we determined whether the myeloid leukemia cells exhibiting CaMKIIγ autophosphorylation expressed any unusual CaMKIIγ isoform(s). An RT-PCR analysis utilizing primers flanking this alternatively spliced CaMKIIγ variable region (FIG. 8C) indicated that in normal CD34+ cells as well as in leukemia cells the previously described CaMKIIγ1 and γ3 isoforms were predominantly and commonly expressed (FIG. 8C). In addition we determined whether the activation (autophosphorylation) of CaMKIIγ observed in the myeloid leukemia cell lines was associated with any particular mutations within the CaMKIIγ coding region in these cells. We PCR—amplified and sequenced the entire CaMKIIγ coding region cDNA from the HL60 and NB4 myeloid cell lines. The CaMKIIγ cDNA coding sequence from both these cell lines matched the GenBank CaMKIIγ1 and γ3 sequences. Thus the CaMKIIγ activation exhibited by the myeloid leukemia cells is not associated with the expression of any aberrantly spliced CaMKIIγ isoform(s) nor with any particular CaMKIIγ coding region mutation(s).

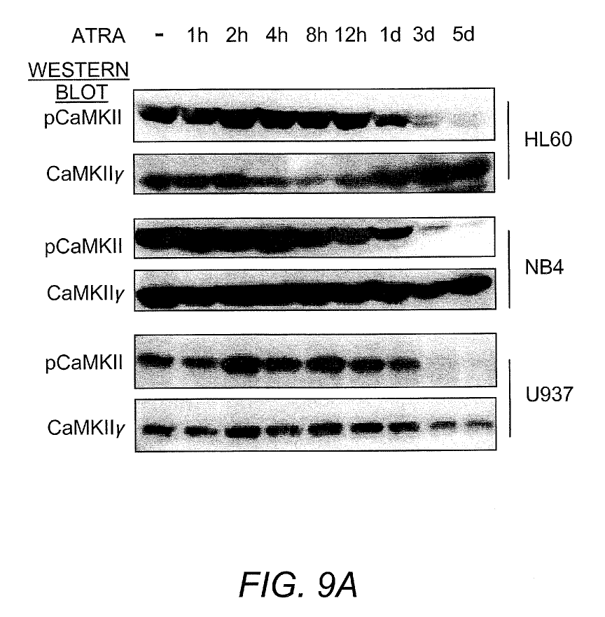

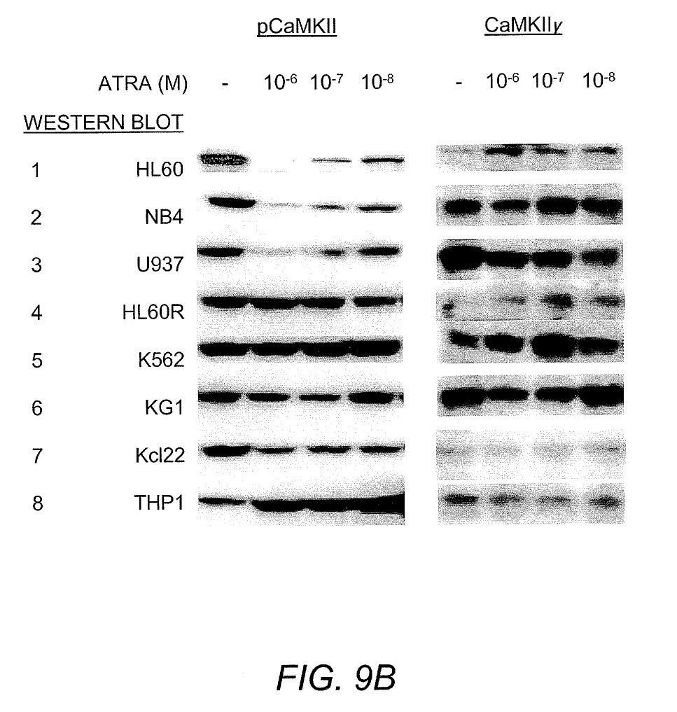

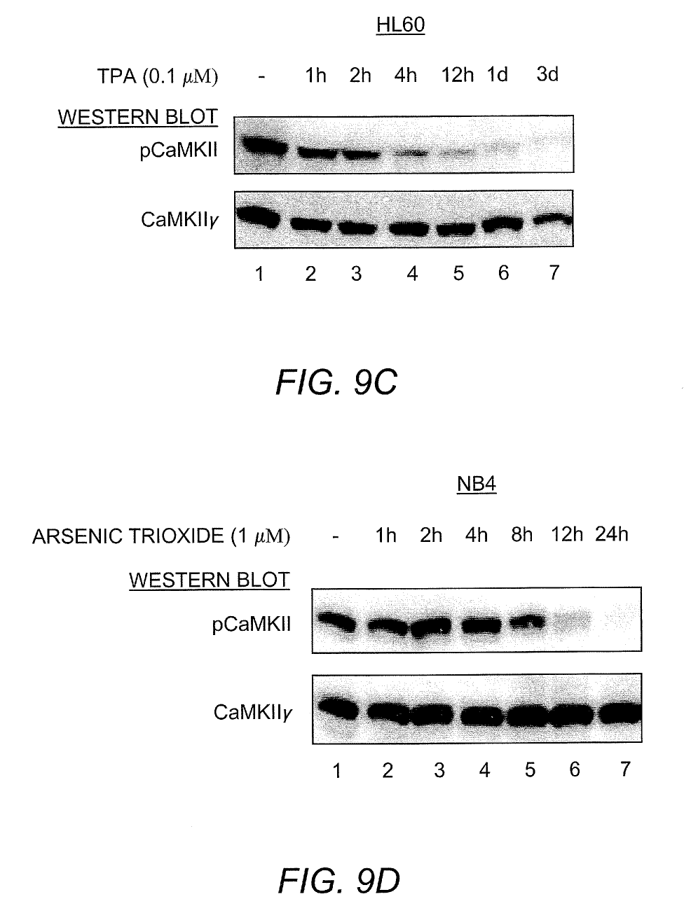

Terminal differentiation/apoptosis of myeloid leukemia cell lines is associated with decreased CaMKIIγ activation. All-trans retinoic acid (ATRA) induces the terminal granulocytic differentiation of certain mycloid leukemia cell lines including HL60 and NB4, and ATRA also induces terminal monocytic differentiation of the U937 cell line (Breitman T et al., Proc Natl Acad Sci USA 77: 2936-2940 (1980); Lanotte M et al., Blood 77: 1080-6 (1991); Olsson I L et al., Cancer Research 42: 3924-3927 (1982)). We utilized Western blots to compare CaMKIIγ expression in these cells at different times after this ATRA-induced terminal differentiation. We observed in all three of these cell lines that ATRA-induced terminal differentiation was associated with a marked decrease in the levels of activated CaMKIIγ, while total CaMKIIγ levels remain essentially unchanged (FIG. 9A). This decrease in activated CaMKIIγ was most prominent 3-5 days post differentiation induction which, as previously observed, is the time that these cells undergo terminal differentiation and cease to proliferate. This ATRA-induced decrease in activated CaMKIIγ was not associated with any decrease in cellular calmodulin levels (not shown). This decreased CaMKIIγ activation was ATRA-dose dependent (FIG. 9B, rows 1-3) and appeared to closely correlate with differentiation induction since a similar decrease was not observed in ATRA-treated HL60R cells, which harbor an inactivating point mutation in RARα that renders them unresponsive to ATRA—induced granulocytic differentiation (Robertson K et al., Blood 80:1885-1889 (1992)) (FIG. 9B, row 4). Similarly other myeloid leukemia cell lines that are insensitive to ATRA-induced differentiation including K562, KG1, THP1, and KCL22 did not exhibit any decrease in activated CaMKIIγ expression following ATRA induction (FIG. 9B, rows 5-8). A similar marked decrease in activated CaMKIIγ was also observed in HL60 cells treated with phorbol myristate acetate (TPA), which induces terminal macrophage-like differentiation of these cells (Rovera G, et al., Proc Natl Acad Sci USA 76: 2779-83 (1979)) (FIG. 9C). Similarly we observe that the terminal differentiation/apoptosis induced by arsenic trioxide in promyelocytic leukemia cell lines such as NB4 (Chen G Q, et al., Blood 88:1052-61 (1996)) is accompanied by a downregulation in phosphorylated CaMKII (FIG. 9D). Thus the terminal differentiation of myeloid leukemia cells is associated with a marked downregulation of the autophosphorylated CaMKIIγ.

Oncogenic bcr-abl regulates CaMKIIγ activation. In contrast to its lack of response to ATRA, the K562 cell line, derived from a patient with CML, undergoes proliferative arrest following exposure to imatinib (gleevec), which is a potent inhibitor of the tyrosine kinase activity of the bcr-abl oncogene. We observed that this imatinib-induced proliferation arrest is accompanied by a rapid, marked decrease in autophosphorylated CaMKIIγ, while there was no significant change in the total levels of CaMKIIγ following this imatinib exposure (FIG. 0A).

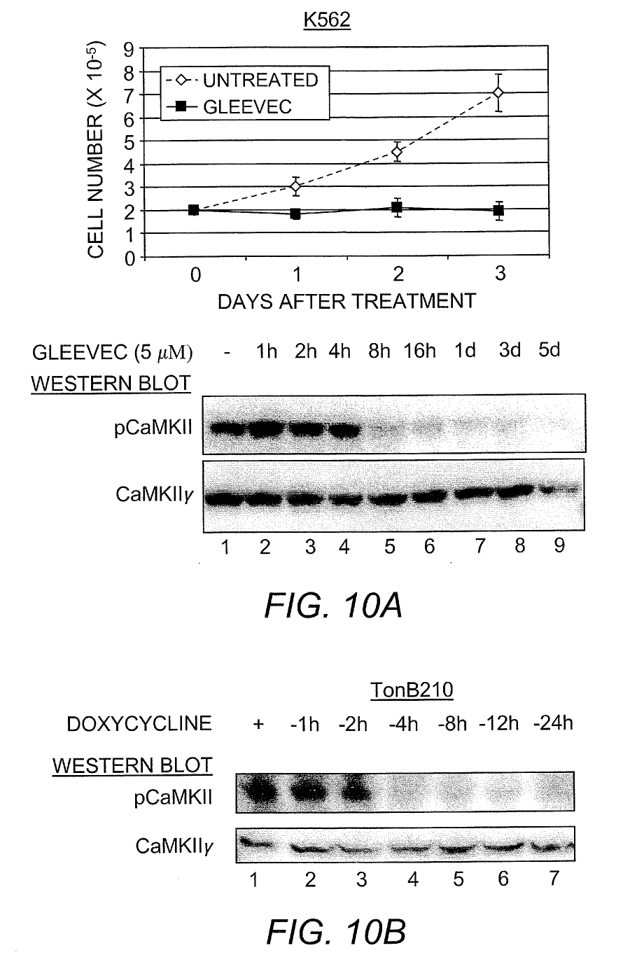

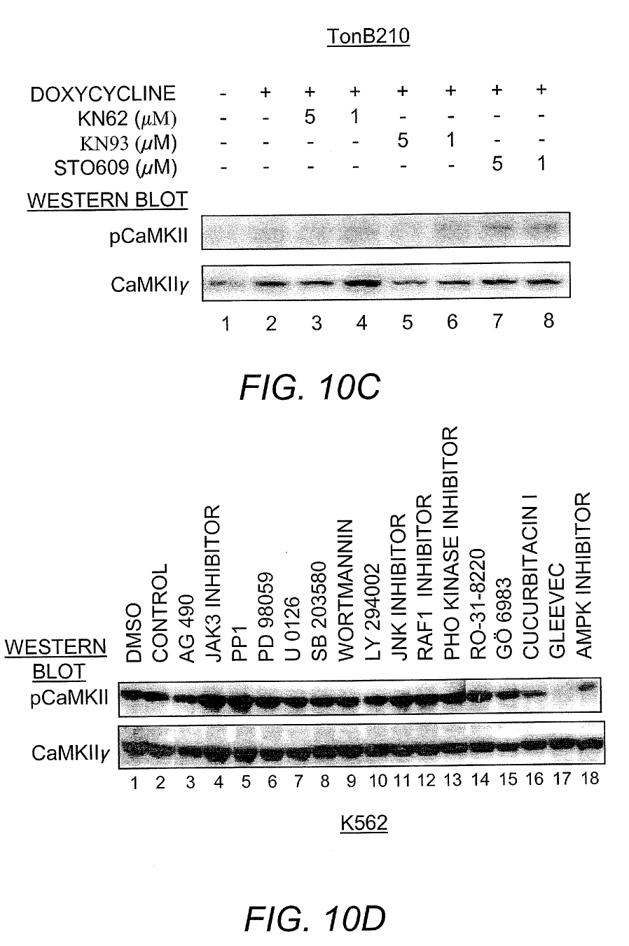

The marked downregulation of CaMKII activation (autophosphorylation) by the bcr-abl inhibitor, imatinib observed in K562 cells suggests that the activation of CaMKIIγ in these CML cells requires bcr-abl activity. To confirm this we utilized the TonB210.1 cell line, which is a Baf3-derived line whose proliferation/viability is dependent upon either IL-3 alone or the tet-regulated expression of bcr-abl (tet-on) (Klucher K M, et al., Blood 91: 3927-34 (1998)). We observe in the TonB210.1 cells that the decreased bcr-abl expression associated with tet withdrawal is also accompanied by a marked and relatively rapid (within 4 hours) reduction in the levels of activated (autophosphorylated) CaMKII, while total CaMKIIγ levels remain unchanged (FIG. 10B). To determine if enhanced bcr-abl expression can initiate CaMKII activation, we first deprived the IL-3 dependent TonB210.1 cells of IL-3 for 24 hours, which induced cell proliferative arrest associated with decreased autophosphorylated CaMKIIγ (FIG. 10C, lane 1). Treatment of these cells with tet restores CaMKII autophosphorylation (FIG. 10C, compare lanes 1 and 2), and this phosphorylation was inhibited in a dose dependent manner by the CaMK chemical inhibitors KN62 and KN93 (FIG. 10C, lanes 3-6), which interfere with the binding of the Ca⁺⁺/calmodulin complex to the CaMKs (Sumi M, et al., Biochem Biophys Res Commun 181: 968-75 (1991)). In contrast this bcr-abl mediated enhancement of CaMKIIγ phosphorylation was not inhibited by the CaM kinase kinase (CaMKK) chemical inhibitor STO-609 (FIG. 10C, lanes 7-8). Taken together these observations with the K562 and TonB210.1 cells indicate that oncogenic bcr-abl directly or indirectly activates CaMKIIγ and that this bcr-abl induced CaMKIIγ activation is dependent on Ca⁺⁺/calmodulin binding to CaMKIIγ.

Effect of inhibitors of different signal transduction pathways on CaMKIIγ activation. The above observations indicate that CaMKII activation is downstream of bcr-abl, but we don't know whether this oncogene directly or indirectly (through one or more of its downstream effectors) mediates this activation. The molecular basis for bcr-abl mediated cell transformation is complex and involves a number of specific downstream signal transduction pathways activated by bcr-abl including Stat5 (de Groot R P et al., Blood 94: 1108-12 (1999)), Ras/Raf1/MAPK (de Groot R P et al., Blood 94: 1108-12 (1999)), PI3 Kinase (Skorski T, et al., Embo J 16: 6151-61 (1997)) and SRC-family kinases (Hu Y et al., Nat Genet. 36:453-61 (2004)). Indeed the cooperative activation of these pathways may be necessary for full bcr-abl mediated transformation (Sonoyama J et al., J Biol Chem 277: 8076-82 (2002)). We utilized specific chemical inhibitors to determine whether any of these signal transduction pathways downstream of bcr-abl might be involved in the bcr-abl mediated activation of CaMKIIγ in K562 cells. In contrast with the gleevec induced downregulation of CaMKIIγ activation (FIG. 10D, lane 17), none of these chemical inhibitors reduced CaMKIIγ activation including those which target Jak/Stat (FIG. 10D, lanes 3,4,16), PI3 kinase (lanes 9,10), p38 (lane 8), JNK, (lane 11), Ras/Raf/MEK/ERK (lanes 6,7,12), PKC (lanes 14,15), AMP kinase (lane 18) and SRC-family kinases (lane 5). Thus the bcr-abl mediated activation of CaMKIIγ does not appear to involve any of the signal transduction pathways that are known downstream effectors of bcr-abl.

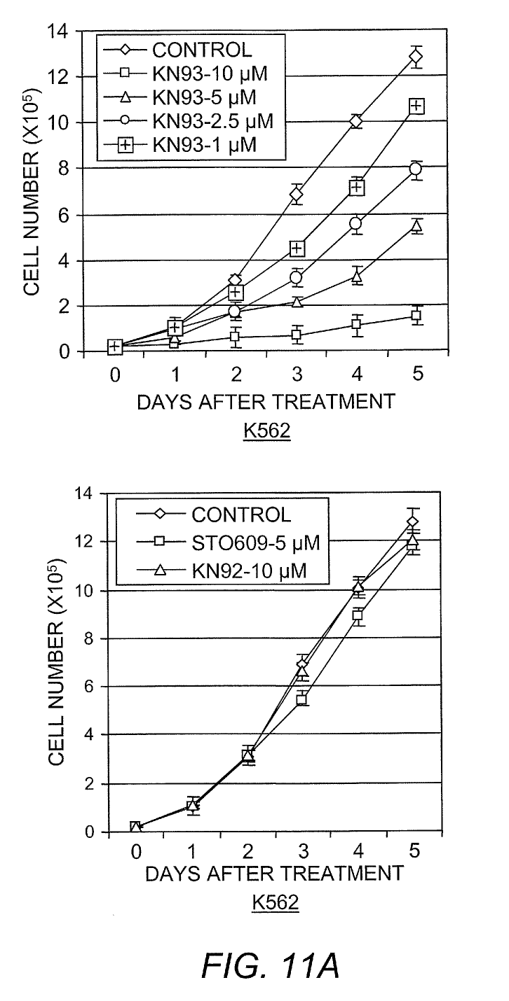

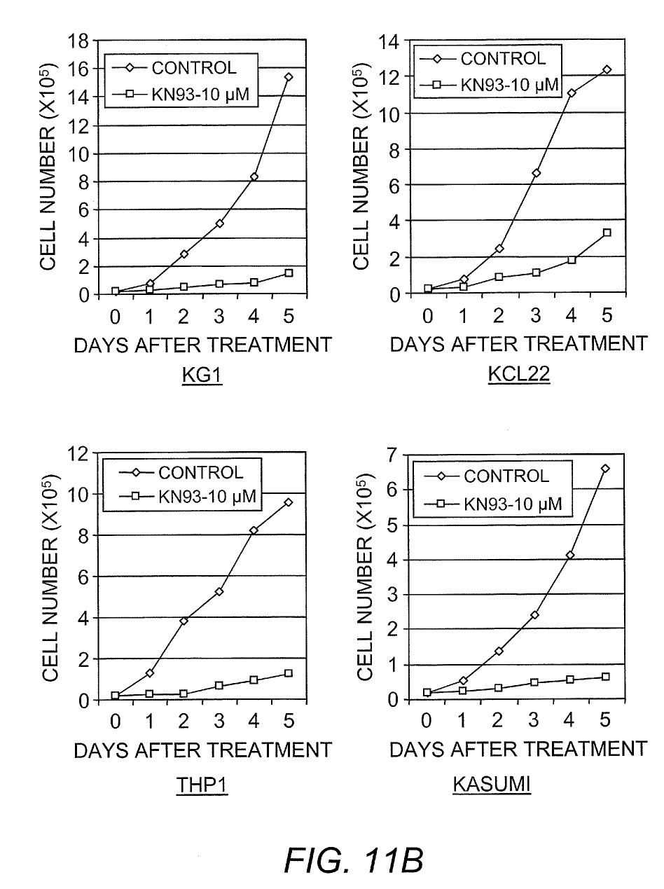

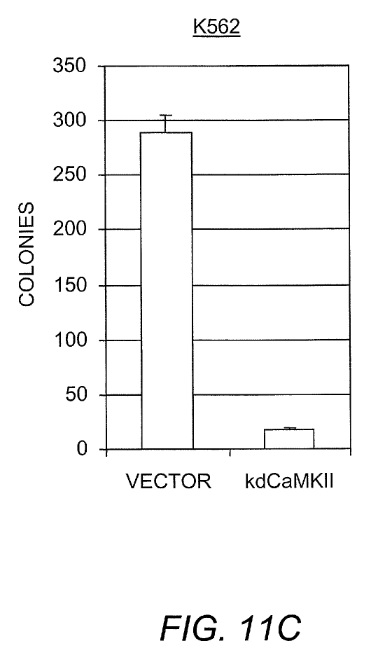

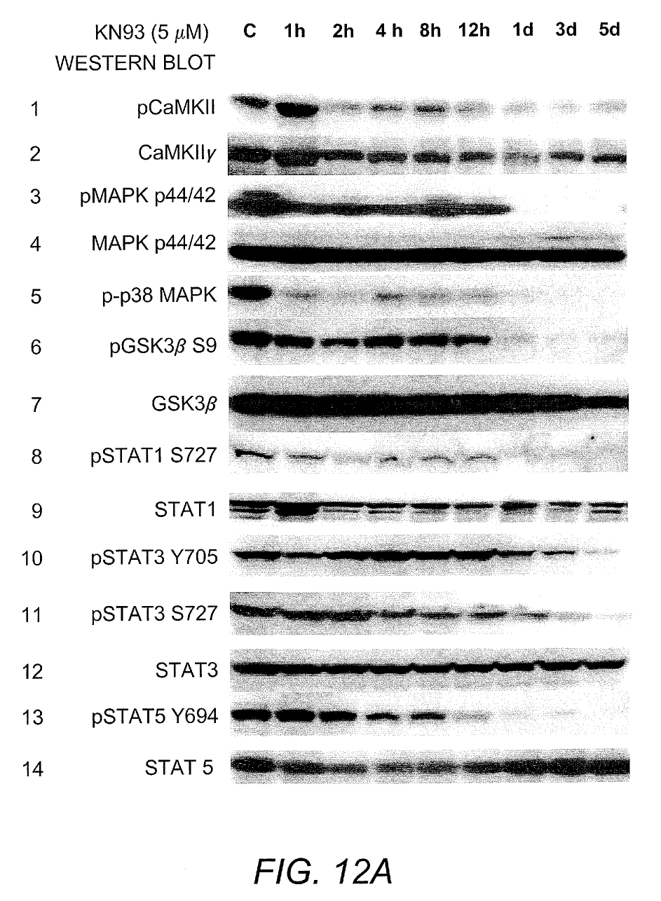

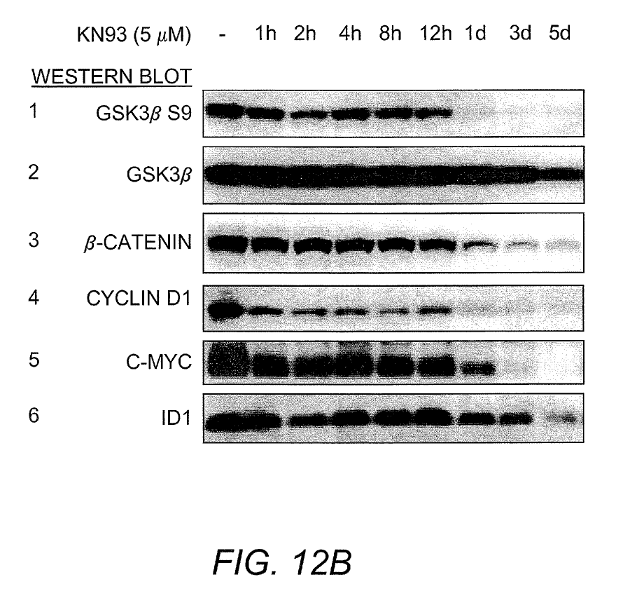

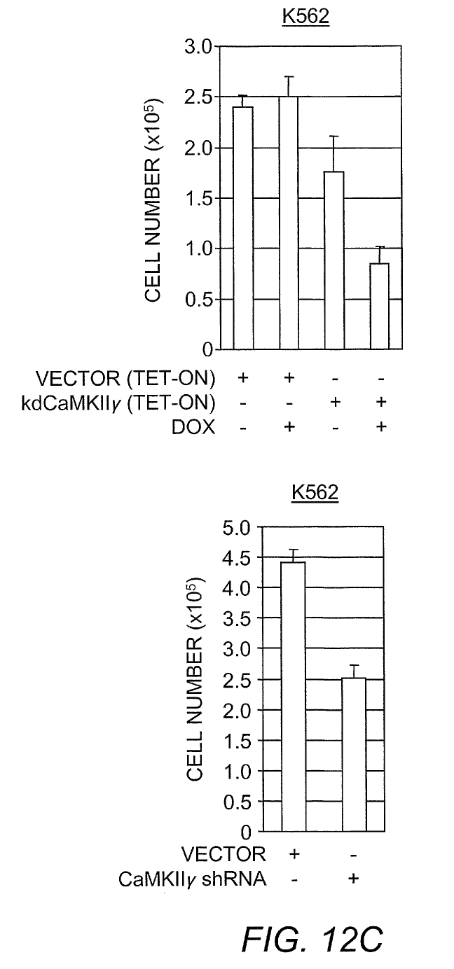

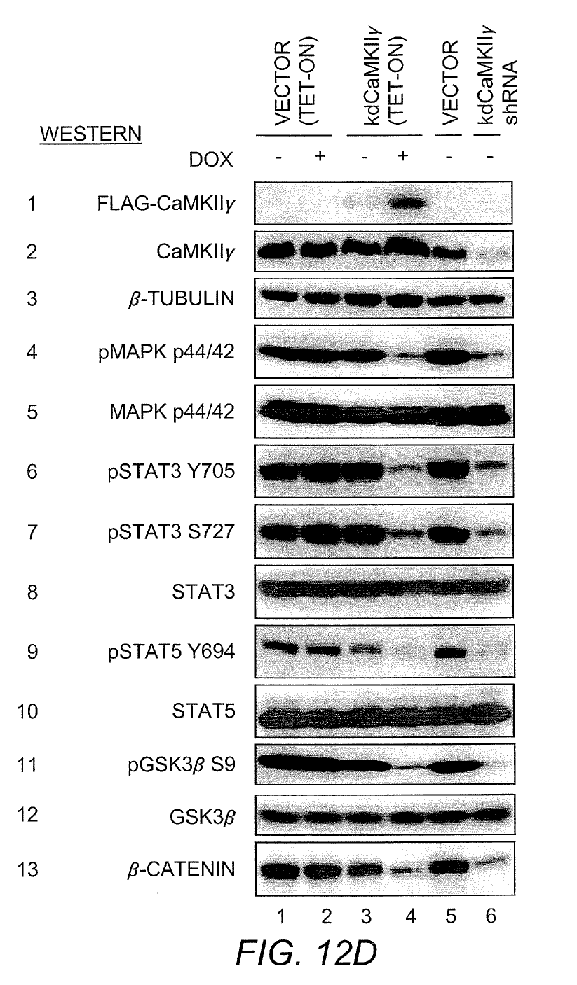

Inhibitors of CaMKII inhibit the proliferation of myeloid leukemia cell lines. To determine whether inhibiting CaMKIIγ activity might have any effect on myeloid leukemia cell proliferation/viability, we treated K562 cells with the CaMK inhibitor KN93. We found that KN93 inhibited the proliferation of K562 cells in a dose dependent manner (FIG. 11A, i). In contrast STO609, a potent small molecule inhibitor of CaMKinase Kinase (CaMKK)(40) did not significantly inhibit K562 proliferation nor did KN92, an inactive structural analog of KN62 (FIG. 11A, ii). The KN93 induced inhibition of K562 cell proliferation was accompanied by a reduction in the levels of the activated (autophosphorylated) CaMKIIγ (FIG. 12A, row 1). KN93 did not affect K562 viability, and no effect on the morphologic differentiation of these cells was observed. A similar KN93-mediated inhibition of cell proliferation without any significant effect on cell viability or morphologic differentiation was also noted in other cultured myeloid leukemia cell lines including KG-1, KCL22, THP1, and Kasumi (FIG. 11B).

To further assess the role of CaMKIIγ in regulating myeloid leukemia cell proliferation, we transiently transfected K562 cells with an expression vector harboring a truncated CaMKIIγ construct mutated at Lys⁴³, a conserved residue near the ATP-binding site of this enzyme. This mutation results in a ‘kinase dead’ enzyme (Hanson P I, et al., Neuron 12: 943-56 (1994)), and expression of this mutant inhibits endogenous CaMKII activity in a dominant negative manner (Kuhl M, et al., J Biol Chem 275: 12701-11 (2000)). We observed a marked reduction in K562 colony formation in cells transfected with this mutated CaMKIIγ compared with cells transfected with vector alone (FIG. 11C). Thus both pharmacological and dominant negative construct inhibition of CaMKII results in inhibition of myeloid leukemia cell proliferation.

Pharmacologic, dominant negative or shRNA-mediated inhibition of CaMKIIγ is associated with the downregulation of multiple signal transduction pathways. We utilized phosphorylation specific antibodies to determine whether the KN93-induced inhibition of K562 cell proliferation (FIG. 11A, i) was associated with any changes in specific phosphoprotein signal transduction pathways. As noted above KN93 treatment of K562 cells is associated with a time dependent reduction in CaMKIIγ phosphorylation (FIG. 12A, row 1). This reduction was associated with a decrease in the activation (phosphorylation) of a number of different critical components of signal transduction pathways including pMAPK p44/42 (FIG. 12A, row 3), p38 (row 5), pStat3 (Y705, S727) (rows 10,11) and pStat5 (Y694) (row 13). These changes induced by KN93 were not accompanied by any change in the total levels of these phosphorylated proteins (FIG. 12A). These observations indicate that in K562 myeloid cells activated CaMKIIγ is involved in regulating the activation (phosphorylation) of MAPK p44/42, Stat3/Stat5, and p38.

We also observe that the KN93 induced downregulation of CaMKII activation is associated with a marked reduction in GSK3β Ser9 phosphorylation (FIG. 12A, row 6). This is of particular interest since GSK3β, a serine/threonine kinase, phosphorylates and promotes the degradation of β-catenin, a key component of the Wnt signaling pathway, which is frequently activated in myeloid leukemia (Cohen P, Frame S, Nat Rev Mol Cell Biol; 2: 769-76 (2001); Simon M et al., Oncogene 24: 2410-20 (2005)). Since phosphorylation of GSK3β at Ser9 inhibits the activity of this enzyme (Eldar-Finkelman H, et al., Proc Natl Acad Sci USA 93:10228-33 (1996)), we would predict that the KN93 induced reduction in GSK3β Ser9 phosphorylation would lead to enhanced GSK3β enzyme activity associated with enhanced degradation of β-catenin. Indeed a marked reduction in the expression of β-catenin is observed in the KN93 treated K562 cells (FIG. 12B, row 3). Consistent with this, in the KN93 induced cells we also observe a reduction of c-myc and cyclin D1 protein levels, both of whose expression is normally upregulated by β catenin (He T C, et al., Science 281:1509-12 (1998); Tetsu O, et al., Nature 398: 422-6 (1999) (FIG. 5B, rows 4,5).

KN93 inhibits CaMKII activity by blocking its interaction with the Ca⁺⁺/calmodulin complex. However, this compound can potentially inhibit other enzymes activated by Ca⁺⁺/calmodulin including CaMKI, CaMKIV, CKLiK as well as the CaMK-kinases (CaMKKs). Nevertheless, several lines of evidence indicate that the effects of KN93 are mediated through CaMKII inhibition rather than through inhibiting any of these other CaMkinases. First, CaMKIV is not expressed in K562 cells (data not shown)), and the CaMKI expressed by K562 is primarily in the inactive (unphosphorylated) form (data not shown). Second, there is little if any CKLiK (CaMKIδ) expression in K562 cells compared with other cell types (data not shown)). Moreover, STO609, a potent small molecule inhibitor of the CaMKKs, exhibits minimal effects on K562 proliferation (FIG. 11A, ii) indicating that neither the CaMKKs nor their downstream substrate, CaMKI are the targets of KN93. In addition we transduced a FLAG-tagged Lys⁴³ mutated, dominant negative CaMKIIγ under control of a doxycycline inducible promoter (designated kdCaMKIIγ (Tet-on)) into K562 cells. Treatment of these cells with doxycycline upregulated expression of this construct (FIG. 12D, row 1, lane 4), and this induced expression was temporally associated with inhibition of K562 proliferation (FIG. 12C, i) together with downregulation of the activation (phosphorylation) of the MAPkinase, Stat3/Stat5 and β-catenin pathways (FIG. 12D, compare lanes 3 vs. 4). Finally we utilized an siRNA approach to knockdown the expression of CaMKIIγ in K562 cells. The shRNA transduced K562 cells with reduced expression of CaMKIIγ (FIG. 12D, row 2, compare lanes 5 vs. 6) exhibited reduced cell proliferation (FIG. 12C, ii), and this is associated with downregulation of the MAPkinase, Jak/Stat and GSK3β/β-catenin pathways (FIG. 12D, compare lanes 5 and 6). Thus the effects of KN93 on K562 cells are likely mediated through its inhibitory activity on CaMKIIγ rather than through any of the other CaMkinases.

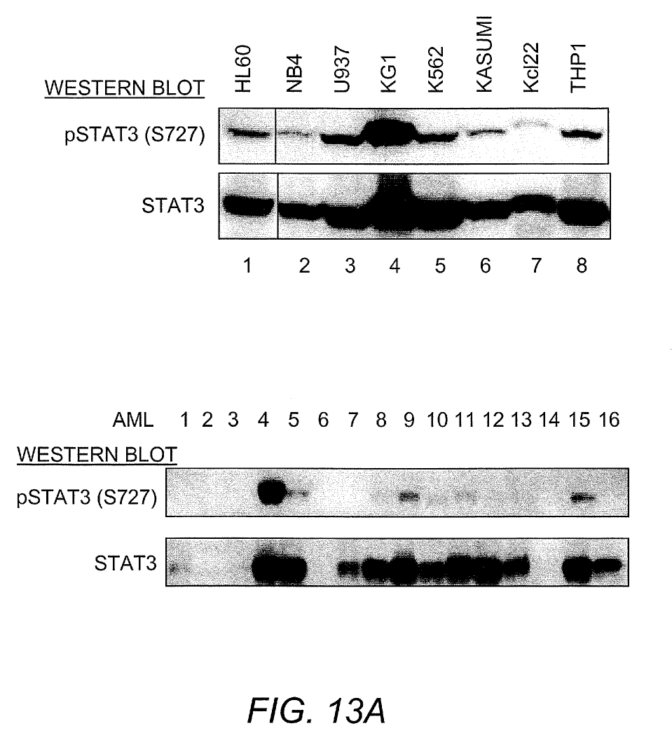

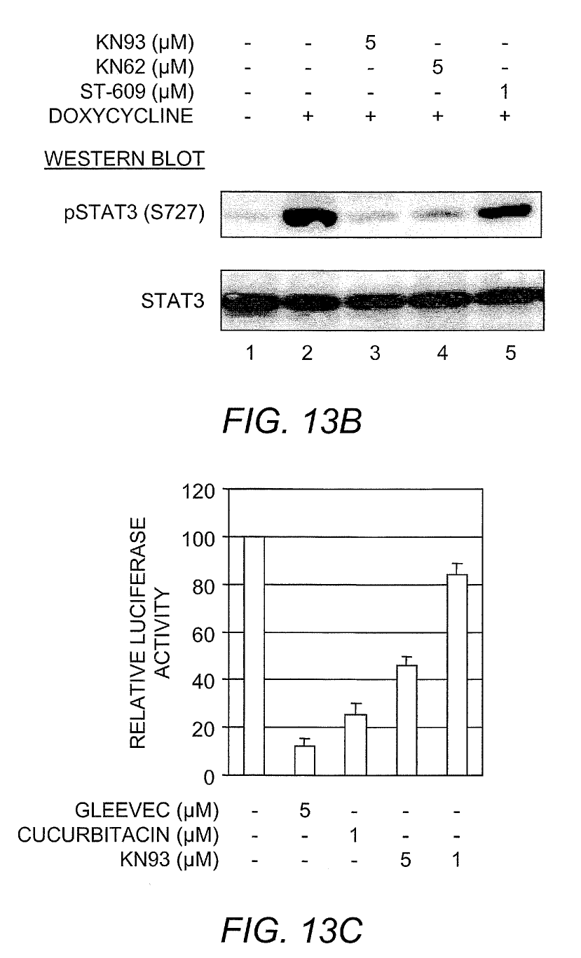

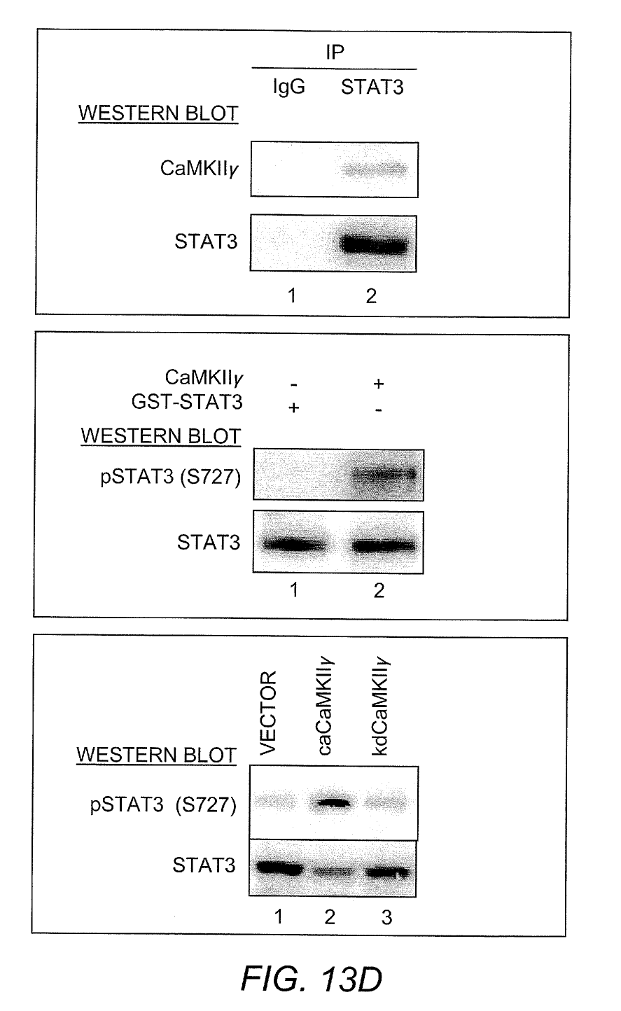

CaMKIIγ directly phosphorylates and activates Stat3 in myeloid leukemia cells. In addition to tyrosine phosphorylation of Stat3 at Y705, Stat3 Ser727 phosphorylation is required for full Stat3 transcriptional activity (Shen Y, et al., Mol Cell Biol 24: 407-19 (2004)). The activated Stat3 has oncogenic activity (Bromberg J F, et al., Cell 98: 295-303 (1999)), and Stat 3 activation is frequently observed in myeloid leukemia cell lines (FIG. 6A, i) as well as myeloid leukemia patient samples (Steensma D P, et al., Leukemia 20: 971-8 (2006)) (FIG. 13A, ii). Our observation that the CaMKII inhibitor KN93 induces a decrease in Stat3 Ser727 phosphorylation (FIG. 12A, row 11) suggests that Stat3 may be a direct substrate of CaMKIIγ in myeloid leukemia cells. Consistent with this we observe that 9/11 of the primary AML samples that exhibit CaMKIIγ activation (FIG. 8B) also exhibit Stat3 Ser727 phosphorylation, while none of the five primary AML samples that were negative for CaMKIIγ activation exhibit Stat3 Ser 727 phosphorylation (FIG. 13A, ii). In addition we observe in the TonB210 cells that the tet-induced bcr-abl induction is associated with a marked increase in Stat3 Ser727 phosphorylation without any change in total Stat3 levels (FIG. 13B, compare lanes 1 vs 2). Moreover, this enhanced Stat3 Ser727 phosphorylation is blocked by KN62 or KN93 (FIG. 13B, lanes 3, 4) indicating that a CaM kinase is involved in this bcr-abl induced Stat3 Ser727 phosphorylation. We also observe that cucurbitacin I, a small molecule Stat3 inhibitor, will, as expected, inhibit Stat3 responsive reporter activity, and this reporter activity is also inhibited by KN93 in a dose dependent manner (FIG. 13C). These latter observations indicate a functional interaction between Stat3 and CaMKII in myeloid leukemia cells.

To further assess the CaMKIIγ/Stat3 interaction we observed that in K562 cells Stat3 co-immunoprecipitates with CaMKIIγ (FIG. 13D, i). Moreover, CaMKIIγ phosphorylates Stat3 at Ser727 both in vitro (FIG. 13D, ii) and in vivo (FIG. 13D, iii). Together these observations suggest that Stat3 is likely a direct substrate of CaMKIIγ in myeloid leukemia cells and that the KN93 induced inhibition of K562 cell proliferation (FIG. 11A) may be mediated, at least in part, through its inhibition of Stat3 Ser727 phosphorylation.

The foregoing is illustrative of the present invention, and not to be construed as limiting thereof. The invention is defined by the following claims, with equivalents of the claims to be included therein.

That which is claimed is:

1. A method of treating cancer in a subject in need thereof, comprising administering said subject a CaM kinase II gamma inhibitor in a treatment effective amount.

2. The method of claim 1, wherein said cancer is selected from the group consisting of acute myelogenous leukemia, chronic myelogenous leukemia, gastrointestinal stromal tumor, small cell lung cancer, non-small cell lung cancer, ovarian cancer, melanoma, mastocytosis, germ cell tumors, pediatric sarcomas, breast cancer, colorectal cancer, pancreatic cancer, prostate cancer.

3. The method of claim 1, wherein said cancer is myeloid leukemia.

4. The method of claim 1, wherein said subject is a mammalian subject.

5. The method of claim 1, wherein said subject is a human subject.

6. The method of claim 1, wherein said CaMKII inhibitors is selected from the group consisting of calcium chelators, calmodulin antagonists, small peptides based on CaMKII protein sequence, nucleic acid-based inhibitors, and mixtures thereof.

7. The method of claim 1, wherein said CaMKII inhibitor is selected from the group consisting of KN62, KN93, H89, HA1004, HA1077, autocamtide-2 related inhibitory peptide or a myristoylated form thereof, K-252a, staurosporine, lavendustin C, BAPTA tetrasodium salt, 5,5′-dibromo-BAPTA, tetrasodium salt, BAPTA/AM, 5,5′-difluoro-BAPTA/AM, EDTA tetrasodium salt, EGTA, EGTA/AM, MAPTAM, TPEN, calmidazolium chloride, calmodulin binding domain, chlorpromazine, Compound 48/80, fluphenazine-N-2-chloroethane dihydrochloride, melittin, ophiobolin A, pentamidine isethionate, phenoxybenzamine, trifluoperazine, W-5, W-7, W-12, W-13, CaM kinase II 290-309, [Ala286]CaMKII Inhibitor 281-301, CaMKII Inhibitor 281-309, and mixtures thereof.

8. A method of treating a cancer in a subject in need thereof, comprising concurrently administering said subject a retinoid and a CaM kinase II (CaMK II) inhibitor,
said CaMK II inhibitor administered in an amount effective to enhance the activity of said retinoid; and
said retinoid administered in an amount effective to treat said cancer.

9. The method of claim 8, wherein said retinoid and said CaMK II inhibitor are administered in a synergistically effective amount.

10. The method of claim 8, wherein said CaMK II is CaMK II gamma.

11. The method of claim 8, wherein said CaMK II inhibitor and said retinoid are administered simultaneously or sequentially.

12. The method of claim 8, wherein said cancer is selected from the group consisting of acute myelogenous leukemia, chronic myelogenous leukemia, gastrointestinal stromal tumor, small cell lung cancer, non-small cell lung cancer, ovarian cancer, melanoma, mastocytosis, germ cell tumors, pediatric sarcomas, breast cancer, colorectal cancer, pancreatic cancer, prostate cancer.

13. The method of claim 8, wherein said cancer is myeloid leukemia.

14. The method of claim 8, wherein said subject is a mammalian subject.

15. The method of claim 8, wherein said subject is a human subject.

16. The method of claim 8, wherein said CaMKII inhibitors is selected from the group consisting of calcium chelators, calmodulin antagonists, small peptides based on CaMKII protein sequence, nucleic acid-based inhibitors, and mixtures thereof.

17. The method of claim 8, wherein said CaMKII inhibitor is selected from the group consisting of KN62, KN93, H89, HA1004, HA1077, autocamtide-2 related inhibitory peptide or a myristoylated form thereof, K-252a, staurosporine, lavendustin C, BAPTA tetrasodium salt, 5,5′-dibromo-BAPTA, tetrasodium salt, BAPTA/AM, 5,5′-difluoro-BAPTA/AM, EDTA tetrasodium salt, EGTA, EGTA/AM, MAPTAM, TPEN, calmidazolium chloride, calmodulin binding domain, chlorpromazine, Compound 48/80, fluphenazine-N-2-chloroethane dihydrochloride, melittin, ophiobolin A, pentamidine isethionate, phenoxybenzamine, trifluoperazine, W-5, W-7, W-12, W-13, CaM kinase II 290-309, [Ala286]CaMKII Inhibitor 281-301, CaMKII Inhibitor 281-309, and mixtures thereof.

18. The method of claim 8, wherein said retinoid is retinoic acid or a derivative thereof.

19. A pharmaceutical composition comprising, in combination, a CaMK II inhibitor and a retinoid.

20. The composition of claim 19, wherein said CaMK II inhibitor and said retinoid are included in said composition in a synergistically effective amount.

21. The composition of claim 19, wherein said CaMK II inhibitors is selected from the group consisting of calcium chelators, calmodulin antagonists, small peptides based on CaMKII protein sequence, nucleic acid-based inhibitors, and mixtures thereof.

22. The composition of claim 19, wherein said CaMKII inhibitor is selected from the group consisting of KN62, KN93, H89, HA1004, HA1077, autocamtide-2 related inhibitory peptide or a myristoylated form thereof, K-252a, staurosporine, lavendustin C, BAPTA tetrasodium salt, 5,5′-dibromo-BAPTA, tetrasodium salt, BAPTA/AM, 5,5′-difluoro-BAPTA/AM, EDTA tetrasodium salt, EGTA, EGTA/AM, MAPTAM, TPEN, calmidazolium chloride, calmodulin binding domain, chlorpromazine, Compound 48/80, fluphenazine-N-2-chloroethane dihydrochloride, melittin, ophiobolin A, pentamidine isethionate, phenoxybenzamine, trifluoperazine, W-5, W-7, W-12, W-13, CaM kinase II 290-309, [Ala286]CaMKII Inhibitor 281-301, CaMKII Inhibitor 281-309, and mixtures thereof.

23. The composition of claim 19, wherein said retinoid is retinoic acid or a derivative thereof.
